(19) United States
(12) Patent Application Publication
(10) Pub. No.: US 2020/0386770 A1
(43) Pub. Date: Dec. 10, 2020

(54) MONOCLONAL ANTIBODIES AGAINST HUMAN PD-L1 AND USES THEREOF

(71) Applicant: YUROGEN BIOSYSTEMS LLC, Worcester, MA (US)

(72) Inventors: Yang LOU, Worcester, MA (US); Hai WU, Worcester, MA (US)

(21) Appl. No.: 16/891,862

(22) Filed: Jun. 3, 2020

Related U.S. Application Data
(60) Provisional application No. 62/857,145, filed on Jun. 4, 2019.

(51) Int. Cl. G01N 33/68; G01N 33/533; G01N 33/543; C07K 16/28

(52) U.S. Cl. CPC G01N 33/6854; G01N 33/533; G01N 33/543; C07K 2317/567; C07K 2317/565; C07K 2317/24; C07K 16/2827

(57) ABSTRACT

The disclosure provides an anti-human PD-L1 antibody and uses thereof. The anti-human PD-L1 antibody includes: a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, and conservative modifications thereof; a V_{H} CDR2 selected from the group consisting SEQ ID NO: 7, SEQ ID NO: 8, SEQ ID NO: 9, SEQ ID NO: 10, SEQ ID NO: 11, SEQ ID NO: 12, and conservative modifications thereof; a V_{H} CDR3 selected from the group consisting of SEQ ID NO: 13, SEQ ID NO: 14, SEQ ID NO: 15, SEQ ID NO: 16, SEQ ID NO: 17, SEQ ID NO: 18, and conservative modifications thereof; a V_{L} CDR1 selected from the group consisting SEQ ID NO: 19, SEQ ID NO: 20, SEQ ID NO: 21, SEQ ID NO: 22, SEQ ID NO: 23, SEQ ID NO: 24, and conservative modifications thereof; a V_{L} CDR2 selected from the group consisting of SEQ ID NO: 25, SEQ ID NO: 26, SEQ ID NO: 27, SEQ ID NO: 28, SEQ ID NO: 29, SEQ ID NO: 30, and conservative modifications thereof; and a V_{L} CDR3 selected from the group consisting of SEQ ID NO: 31, SEQ ID NO: 32, SEQ ID NO: 33, SEQ ID NO: 34, SEQ ID NO: 35, SEQ ID NO: 36, and conservative modifications thereof.

### FIELD

The disclosure relates to an antibody against programmed death ligand-1 (PD-L1). Specifically, the disclosure relates to a rabbit monoclonal antibody against the extracellular domain of human PD-L1 and uses thereof.

### BACKGROUND

Programmed death 1 (PD-1) is a member of the CD28 family of receptors. PD-1 can be expressed on activated T and B cells and activated myeloid cells. PD-1 can elicit inhibitory signals upon co-ligation with the T cell receptor (TCR). PD-1 can play a role in immunoregulation and peripheral tolerance. PD-1 can also be an immune checkpoint and protect against autoimmune responses via induction of apoptosis in antigen-specific T cells and inhibition of apoptosis in regulatory T cells.

PD-1 has two cell surface ligands: PD-L1 and programmed death ligand-2 (PD-L2). PD-L1 and PD-L2 have been shown to down-regulate T cell activation and cytokine secretion upon binding to PD-1. Both PD-L1 and PD-L2 are B7 homologs that specifically bind to PD-1.

PD-L1 is encoded by human CD274 gene, also known as cluster of differentiation 274 (CD274) or B7 homolog 1 (B7-H1). PD-L1 is a type 1 transmembrane glycoprotein and has a molecular weight of 40 kDa. Expression of PD-L1 has been found on surfaces of multiple types of immune cells including activated B and T cells, naive lymphocytes, dendritic cells, and monocytes. Expression of PD-L1 has also been found in several human cancers, including lung, ovarian, and colon carcinoma, and various myelomas. In addition, mRNA of PD-L1 can be detected in non-lymphoid cells such as vascular endothelial cells, epithelial cells, muscle cells, and tonsil cells.

### SUMMARY

Embodiments provide a rabbit antibody against the extracellular domain of human PD-L1 and uses thereof. In an embodiment, the antibody can include: (a) a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, and conservative modifications thereof; (b) a V_{H} CDR2 selected from the group consisting SEQ ID NO: 7, SEQ ID NO: 8, SEQ ID NO: 9, SEQ ID NO: 10, SEQ ID NO: 11, SEQ ID NO: 12, and conservative modifications thereof; (c) a V_{H} CDR3 selected from the group consisting of SEQ ID NO: 13, SEQ ID NO: 14, SEQ ID NO: 15, SEQ ID NO: 16, SEQ ID NO: 17, SEQ ID NO: 18, and conservative modifications thereof; (d) a V_{L} CDR1 selected from the group consisting SEQ ID NO: 19, SEQ ID NO: 20, SEQ ID NO: 21, SEQ ID NO: 22, SEQ ID NO: 23, SEQ ID NO: 24, and conservative modifications thereof; (e) a V_{L} CDR2 selected from the group consisting of SEQ ID NO: 25, SEQ ID NO: 26, SEQ ID NO: 27, SEQ ID NO: 28, SEQ ID NO: 29, SEQ ID NO: 30, and conservative modifications thereof; and (f) a V_{L} CDR3 selected from the group consisting of SEQ ID NO: 31, SEQ ID NO: 32, SEQ ID NO: 33, SEQ ID NO: 34, SEQ ID NO: 35, SEQ ID NO: 36, and conservative modifications thereof.

In an embodiment, the antibody can include: (a) a V_{H} CDR1 including the amino acid sequence of SEQ ID NO: 1 or a conservative modification thereof; (b) a V_{H} CDR2 including the amino acid sequence of SEQ ID NO: 7 or a conservative modification thereof; (c) a V_{H} CDR3 including the amino acid sequence of SEQ ID NO: 13 or a conservative modification thereof; (d) the V_{L} CDR1 including the amino acid sequence of SEQ ID NO: 19 or a conservative modification thereof; (e) a V_{L} CDR2 including the amino acid sequence of SEQ ID NO: 25 or a conservative modification thereof; and (f) a V_{L} CDR3 including the amino acid sequence of SEQ ID NO: 31 or a conservative modification thereof.

In an embodiment, the antibody can include: (a) a V_{H} CDR1 including the amino acid sequence of SEQ ID NO: 2 or a conservative modification thereof; (b) a V_{H} CDR2 including the amino acid sequence of SEQ ID NO: 8 or a conservative modification thereof; (c) a V_{H} CDR3 including the amino acid sequence of SEQ ID NO: 14 or a conservative modification thereof; (d) a V_{L} CDR1 including the amino acid sequence of SEQ ID NO: 20 or a conservative modification thereof; (e) a V_{L} CDR2 including the amino acid sequence of SEQ ID NO: 26 or a conservative modification thereof; and (f) the V_{L} CDR3 including the amino acid sequence of SEQ ID NO: 32 or a conservative modification thereof.

In an embodiment, the antibody can include: (a) a V_{H} CDR1 including the amino acid sequence of SEQ ID NO: 3 or a conservative modification thereof; (b) a V_{H} CDR2 including the amino acid sequence of SEQ ID NO: 9 or a conservative modification thereof; (c) a V_{H} CDR3 including the amino acid sequence of SEQ ID NO: 15 or a conservative modification thereof; (d) a V_{L} CDR1 including the amino acid sequence of SEQ ID NO: 21 or a conservative modification thereof; (e) a V_{L} CDR2 including the amino acid sequence of SEQ ID NO: 27 or a conservative modification thereof; and (f) a V_{L} CDR3 including the amino acid sequence of SEQ ID NO: 33 or a conservative modification thereof.

In an embodiment, the antibody can include: (a) a V_{H} CDR1 including the amino acid sequence of SEQ ID NO: 4 or a conservative modification thereof; (b) a V_{H} CDR2 including the amino acid sequence of SEQ ID NO: 10 or a conservative modification thereof; (c) a V_{H} CDR3 including the amino acid sequence of SEQ ID NO: 16 or a conservative modification thereof; (d) a V_{L} CDR1 including the amino acid sequence of SEQ ID NO: 22 or a conservative modification thereof; (e) a V_{L} CDR2 including the amino acid sequence of SEQ ID NO: 28 or a conservative modification thereof; and (f) a V_{L} CDR3 including the amino acid sequence of SEQ ID NO: 34 or a conservative modification thereof.

In an embodiment, the antibody can include: (a) a V_{H} CDR1 including the amino acid sequence of SEQ ID NO: 5 or a conservative modification thereof; (b) a V_{H} CDR2 including the amino acid sequence of SEQ ID NO: 11 or a conservative modification thereof; (c) a V_{H} CDR3 including the amino acid sequence of SEQ ID NO: 17 or a conservative modification thereof; (d) a V_{L} CDR1 including the amino acid sequence of SEQ ID NO: 23 or a conservative modification thereof; (e) a V_{L} CDR2 including the amino acid sequence of SEQ ID NO:29 or a conservative modification thereof; and (f) a V_{L} CDR3 including the amino acid sequence of SEQ ID NO: 35 or a conservative modification thereof.

In an embodiment, the antibody can include: (a) a V_{H} CDR1 including the amino acid sequence of SEQ ID NO: 6 or a conservative modification thereof; (b) a V_{H} CDR2 including the amino acid sequence of SEQ ID NO: 12 or a conservative modification thereof; (c) a V_{H} CDR3 including the amino acid sequence of SEQ ID NO: 18 or a conservative modification thereof; (d) a V_{L} CDR1 including the amino acid sequence of SEQ ID NO: 24 or a conservative modification thereof; (e) a V_{L} CDR2 including the amino acid sequence of SEQ ID NO: 30 or a conservative modification thereof; and (f) a V_{L} CDR3 including the amino acid sequence of SEQ ID NO: 36 or a conservative modification thereof.

Embodiments also provide an ELISA kit for the detection of expression of human PD-L1. In an embodiment, the ELISA kit can include the rabbit antibody against the extracellular domain of human PD-L1.

Embodiments further provide a method for detecting human PD-L1. In an embodiment, the method can include adding a first antibody that is the rabbit antibody against the extracellular domain of human PD-L1. In an embodiment, the method is a direct ELISA. In an embodiment, the method is a capture ELISA. In an embodiment, the method is a sandwich ELISA. In an embodiment, the method further includes: adding a second antibody including a conjugate for detection, in which the second antibody is different from the first antibody, and the first and second antibodies are each selected from the group consisting of 3B8, 2C5, and 8C6.

### BRIEF DESCRIPTION OF DRAWINGS

FIG. 1 illustrates the structure of a rabbit anti-human PD-L1 mAb, in accordance with an embodiment.

FIG. 2A illustrates sequence alignments of CDR1s of heavy chains of the rabbit anti-human PD-L1 mAbs, in accordance with some embodiments including 2C5, 3B8, 8C6, 10H6, 11G7, and 12H2.

FIG. 2B illustrates sequence alignments of CDR2s of heavy chains of the rabbit anti-human PD-L1 mAbs, in accordance with some embodiments including 2C5, 3B8, 8C6, 10H6, 11G7, and 12H2.

FIG. 2C illustrates sequence alignments of CDR3s of heavy chains of the rabbit anti-human PD-L1 mAbs, in accordance with some embodiments including 2C5, 3B8, 8C6, 10H6, 11G7, and 12H2.

FIG. 3A illustrates sequence alignments of CDR1s of light chains of the rabbit anti-human PD-L1 mAbs, in accordance with some embodiments including 2C5, 3B8, 8C6, 10H6, 11G7, and 12H2.

FIG. 3B illustrates sequence alignments of CDR2s of light chains of the rabbit anti-human PD-L1 mAbs, in accordance with some embodiments including 2C5, 3B8, 8C6, 10H6, 11G7, and 12H2.

FIG. 3C illustrates sequence alignments of CDR3s of light chains of the rabbit anti-human PD-L1 mAbs, in accordance with some embodiments including 2C5, 3B8, 8C6, 10H6, 11G7, and 12H2.

FIG. 4A illustrates sequence alignments of FR1s of heavy chains of the rabbit anti-human PD-L1 mAbs, in accordance with some embodiments including 2C5, 3B8, 8C6, 10H6, 11G7, and 12H2.

FIG. 4B illustrates sequence alignments of FR2s of heavy chains of the rabbit anti-human PD-L1 mAbs, in accordance with some embodiments including 2C5, 3B8, 8C6, 10H6, 11G7, and 12H2.

FIG. 4C illustrates sequence alignments of FR3s of heavy chains of the rabbit anti-human PD-L1 mAbs, in accordance with some embodiments including 2C5, 3B8, 8C6, 10H6, 11G7, and 12H2.

FIG. 4D illustrates sequence alignments of FR4s of heavy chains of the rabbit anti-human PD-L1 mAbs, in accordance with some embodiments including 2C5, 3B8, 8C6, 10H6, 11G7, and 12H2.

FIG. 5A illustrates sequence alignments of FR1s of light chains of the rabbit anti-human PD-L1 mAbs, in accordance with some embodiments including 2C5, 3B8, 8C6, 10H6, 11G7, and 12H2.

FIG. 5B illustrates sequence alignments of FR2s of light chains of the rabbit anti-human PD-L1 mAbs, in accordance with some embodiments including 2C5, 3B8, 8C6, 10H6, 11G7, and 12H2.

FIG. 5C illustrates sequence alignments of FR3s of light chains of the rabbit anti-human PD-L1 mAbs, in accordance with some embodiments including 2C5, 3B8, 8C6, 10H6, 11G7, and 12H2.

FIG. 5D illustrates sequence alignments of FR4s of light chains of the rabbit anti-human PD-L1 mAbs, in accordance with some embodiments including 2C5, 3B8, 8C6, 10H6, 11G7, and 12H2.

FIG. 6 illustrates sequence alignments of variable regions of the heavy chains of the rabbit anti-human PD-L1 mAbs, in accordance with some embodiments including 2C5, 3B8, 8C6, 10H6, 11G7, and 12H2.

FIG. 7 illustrates sequence alignments of variable regions of the light chains of the rabbit anti-human PD-L1 mAbs, in accordance with some embodiments including 2C5, 3B8, 8C6, 10H6, 11G7, and 12H2.

FIG. 8 illustrates sequence alignments of heavy chains of Fab fragments of the rabbit anti-human PD-L1 mAbs, in accordance with some embodiments including 2C5, 3B8, 8C6, 10H6, 11G7, and 12H2.

FIG. 9 illustrates sequence alignments of light chains of Fab fragments of the rabbit anti-human PD-L1 mAbs, in accordance with some embodiments including 2C5, 3B8, 8C6, 10H6, 11G7, and 12H2.

FIG. 10 illustrates sequence alignments of Fc fragments of the rabbit anti-human PD-L1 mAbs, in accordance with some embodiments including 2C5, 3B8, 8C6, 10H6, 11G7, and 12H2.

FIG. 11 shows standard curves of 2C5, 3B8, 8C6, 10H6, 11G7, and 12H2 in a direct ELISA for detecting human PD-L1, in accordance with an embodiment.

FIG. 12 shows results of direct ELISA for detecting human PD-L2 using 2C5, 3B8, 8C6, 10H6, 11G7, and 12H2, in accordance with an embodiment.

FIG. 13 shows standard curves of 2C5, 3B8, 8C6, 10H6, 11G7, and 12H2 in a capture ELISA for detecting human PD-L1, in accordance with an embodiment

FIG. 14 A shows results of sandwich ELISA using the rabbit anti-human PD-L1 mAbs 2C5 as the capture antibody and 8C6 as the detection antibody, in accordance with an embodiment.

FIG. 14B shows results of sandwich ELISA using the rabbit anti-human PD-L1 mAbs 8C6 as the capture antibody and 2C5 as the detection antibody, in accordance with an embodiment.

FIG. 14C shows results of sandwich ELISA using the rabbit anti-human PD-L1 mAbs 2C5 as the capture antibody and 3B8 as the detection antibody, in accordance with an embodiment.

FIG. 14D shows results of sandwich ELISA using the rabbit anti-human PD-L1 mAbs 3B8 as the capture antibody and 2C5 as the detection antibody, in accordance with an embodiment.

FIG. 14E shows results of sandwich ELISA using the rabbit anti-human PD-L1 mAbs 8C6 as the capture antibody and 3B8 as the detection antibody, in accordance with an embodiment.

### DETAILED DESCRIPTION

The disclosure generally relates to an anti-PD-L1 antibody. Specifically, the disclosure relates to a rabbit monoclonal antibody (mAb) against the extracellular domain of human PD-L1 protein and uses thereof.

PD-L1 can be a biomarker and have diagnostic and prognostic values for multiple human tumor types. The interaction of PD-L1 on tumor cell with PD-1 can trigger immuno-inhibition and/or cell death of tumor-specific T cells. The expression level of PD-L1 can correlate with poor prognosis and survival of animals that bear tumors including melanoma, ovary cancer, colorectal cancer, and renal cancer.

The embodiments herein provide rabbit anti-human PD-L1 mAbs and immunoassays that can specifically detect and accurately quantify the expression of PD-L1. The rabbit anti-human PD-L1 mAbs are useful in diagnostic and prognostic measures and in investigating the mechanisms of action of PD-L1 dynamics in immuno-oncology.

The term “antibody” herein can be used in the broadest sense and encompasses various antibody structures, including but not limited to a Y-shaped antibody, namely full-length antibody, an antigen-binding portion of the Y-shaped antibody, and a genetic or chemical modification thereof. The antigen-binding portion refers to one or more portions or fragments of the Y-shaped antibody and can retain the ability of the antibody to bind to PD-L1 specifically. It has been shown that the antigen-binding function of a full-length antibody can be performed by fragments of the full-length antibody.

The term “monoclonal antibody” (mAb) refers to an antibody having a substantially homogeneous population. The individual antibodies of the population are substantially identical, except for possible naturally occurring mutations that may be present in minor amounts. A monoclonal antibody can display a single binding specificity and affinity for a particular epitope on an antigen. In contrast to polyclonal antibodies that typically include different antibodies directed against different epitopes, each monoclonal antibody can target the same or substantially identical epitope on the antigen. The modifier “monoclonal” indicates the character of the antibody as being obtained from a substantially homogeneous population of antibodies, and is not to be construed as requiring the production of the antibody by any particular method. The antibody can be made by various methods. In an embodiment, the monoclonal antibody can be made by the hybridoma method. In an embodiment, the monoclonal antibody can be made by recombinant DNA methods. In an embodiment, the monoclonal antibody can be isolated from phage antibody libraries.

The terms “anti-human PD-L1 mAb” and “mAb against human PD-L1” are used interchangeably and refer to monoclonal antibodies capable of binding human PD-L1 with sufficient affinity so that the antibodies are useful as a detecting, diagnostic, and/or therapeutic agent in targeting human PD-L1. The term “affinity” refers to the strength of the total of non-covalent intermolecular interactions between a single binding site of a molecule (e.g., an antibody) and its binding partner (e.g., an antigen). The intermolecular interactions can include hydrogen bonding, electrostatic interactions, hydrophobic and Van der Waals forces.

The modifier “rabbit” in the term “rabbit antibody”, “rabbit anti-human PD-L1 mAb”, or the like, indicates the complementarity-determining regions (CDRs) of the antibody are derived from rabbit germline immunoglobulin sequences. In an embodiment, the rabbit antibody or rabbit anti-human PD-L1 mAb can encompass antibodies whose CDRs are derived from rabbit germline immunoglobulin sequences. In an embodiment, the rabbit antibody or rabbit anti-human PD-L1 mAbs may encompass antibodies whose CDRs are derived from rabbit germline immunoglobulin sequences, and whose framework regions (FRs) are derived from germline immunoglobulin sequences of another mammalian species, such as a mouse and human. In an embodiment, the rabbit antibody or rabbit anti-human PD-L1 mAb may include antibodies whose FRs and CDRs are derived from rabbit germline immunoglobulin sequences. The terms “rabbit antibody” or “rabbit anti-human PD-L1 mAb” may also encompass antibodies containing amino acid residues not encoded by rabbit germline immunoglobulin sequences, e.g., mutations introduced by random or site-specific mutagenesis in vitro or by somatic mutation in vivo. However, the term “rabbit antibody” or “rabbit anti-human PD-L1 mAb” is not intended to include antibodies whose CDRs are derived from the germline of another mammalian species, such as a mouse.

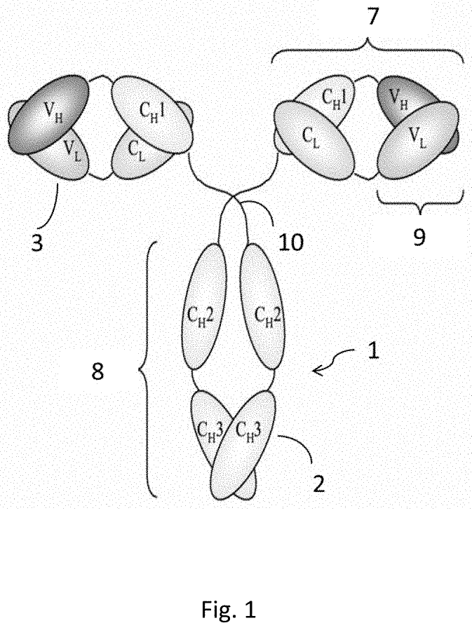

In an embodiment, the rabbit anti-human PD-L1 mAb can be a Y-shaped antibody. Referring to FIG. 1, FIG. 1 illustrates a Y-shaped structure of a rabbit anti-human PD-L1 antibody 1 in accordance with an embodiment. In an embodiment, the rabbit anti-human PD-L1 mAb can include two pairs of heavy chain 2 and light chain 3. The heavy chain 2 can include one variable region (V_{H}) and one or more constant regions (C_{H}s). In an embodiment, the heavy chain 2 can include one V_{H} and three C_{H}s, namely CH1, CH2, and CH3. The V_{H} is closer to the N-terminus of the heavy chain as compared to the three C_{H}s. The V_{H} can exhibit higher variability in the amino acid sequence as compared to the C_{H}s. The V_{H} can differ between different antibodies and can be specific to each antibody. The amino acid sequences of the C_{H}s can be identical across all antibodies of the same isotype (class) but differ between isotypes. The term “isotype” refers to the antibody class (e.g., IgG) that is encoded by the heavy-chain constant-region genes. Mammalian antibodies can have five types of heavy chains: γ, δ, α, μ, and ε. They define classes of antibodies: IgG, IgD, IgA, IgM, and IgE, respectively. Rabbits can have at least four isotypes: IgA, IgE, IgG, and IgM. Humans and mice can have five antibody isotypes: IgA, IgD, IgE, IgG, and IgM.

The light chain 3 can be a small polypeptide subunit relative to the heavy chain 2. The light chain 3 can include one variable region (V_{L}) and one constant (C_{L}) region. The V_{L} is generally at N-terminus of the light chain 3 and exhibits higher variability in amino acid sequence than the C_{L}. The V_{L} can differ between different antibodies and be specific to each antibody in amino acid sequence.

In an embodiment, the variable regions, V_{H} and V_{L}, are responsible for recognizing and binding human PD-L1. In an embodiment, GIs and CL do not directly contact residues of human PD-1.

The two pairs of heavy chain 2 and light chain 3 can form a Y-shaped structure including two Fab (Fragment antigen-binding) fragments 7, one Fc (Fragment crystallizable) fragment 8, and hinge regions 10. The two Fab fragments 7 look like the two arms of the “Y”, and the Fc fragment 8 looks like the base of the “Y”. The hinge regions 10 connect the Fc fragment 8 with the two Fab fragments 7.

Each of the Fab fragments 7 can comprise the V_{H} and CH1 from the heavy chain 2 and the V_{L} and CL from the light chain 3. The Fab fragment 7 contains a variable fragment (Fv fragment) 9 formed of the V_{L} and V_{H}. The Fv fragment 9 accommodates the antigen-binding site, namely paratope. The paratope can be at the tip of the arm of the Y-shaped rabbit mAb 1.

Each of the variable regions, V_{H} and V_{L}, can include complementarity-determining regions (CDRs) and framework regions (FRs). The CDRs are hypervariable regions within the variable regions. The CDRs contain the antigen-contacting residues and are responsible for the ability of the Y-shaped rabbit mAb 1 to recognize and contact the extracellular domain of human PD-L1. The Y-shaped rabbit mAb 1 can include six (6) CDRs, three of which are in the V_{H}, namely V_{H} CDR1, V_{H} CDR2, and V_{H} CDR3, and the other three of which are in the V_{L}, namely V_{L} CDR1, V_{L} CDR3, and V_{L} CDR3.

The CDRs of V_{H} and V_{L} can each be separated by the FRs. The Y-shaped rabbit mAb 1 can contain eight (8) FRs, four of which are in the V_{H} (V_{H} FR1, V_{H} FR2, V_{H} FR3, and V_{H} FR4), and the other four of which are in the V_{L} (V_{L} FR1, V_{L} FR3, and V_{L} FR3, and V_{L} FR4). In an embodiment, the FRs can make up about 85% of the variable region. The term “about” refers to within 10%, preferably within 5%, of a given value or range. The FRs are conserved regions within the variable regions. The FRs can generally act as a scaffold so that the CDRs can adopt three-dimensional structures capable of directly contacting the antigen, i.e., the extracellular domain of human PD-L1.

Among the regions of the Y-shaped rabbit mAb 1, the CDRs determine the specificity and binding affinity of the Y-shaped rabbit mAb 1. The FRs aid in maintaining the overall structure of variable regions of the Y-shaped rabbit mAb 1 and provide structural support to the CDRs, enabling the CDRs to adopt proper conformations for antigen binding.

The three-dimensional structure of the FRs can be conserved across different antibodies.

In an embodiment, the CDRs of the Y-shaped rabbit mAb 1 can be grafted into FRs of another antibody from other species while retaining their ability to bind human PD-L1, forming a mosaic antibody. In an embodiment, the CDRs of the Y-shaped rabbit mAb 1 are grafted into FRs of a human antibody, forming a humanized antibody against human PD-L1.

The Fc fragment 8 can be formed of CH2 and CH3 from the two heavy chains 2. In an embodiment, the Fc fragment 8 can include three constant domains. As the Fc fragment 8 can be composed of the constant domains of the heavy chains, the classes of the heavy chains can be used to categorize the antibody. The Fc fragment 8 of the Y-shaped rabbit mAb 1 generally does not involve binding the antigen. In an embodiment, the Fc fragment 8 can play a role in modulating immune cell activity, for example, by binding to a specific class of Fc receptors or other immune molecules such as complement proteins. In an embodiment, the Fc fragment 8 can play a role in generating an appropriate immune response when the CDRs bind to the antigen. In an embodiment, the Fc fragment 8 can mediate different physiological effects that can include but not limited to recognition of opsonized particles when binding to FcγR, degranulation of mast cells, basophils, and eosinophils when binding to FCC receptors, lysis of cells or complement-dependent cytotoxicity when binding to complement proteins, antibody-dependent cellular cytotoxicity (ADCC), antibody-dependent cell phagocytosis (ADCP), interaction with the neonatal Fc receptor (FcRn) to slow down antibody degradation and extend its serum half-life.

Examples of the Y-shaped rabbit anti-human PD-L1 mAbs can include 2C5, 3B8, 8C6, 10H6, 11G7, and 12H2. These antibodies recognize and bind epitopes on the extracellular domain of human PD-L1. Their structures, antigen-binding portions, derivatives, and applications are described below.

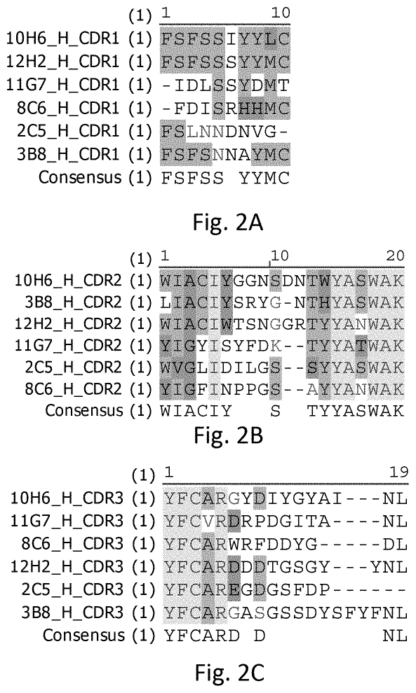

Referring to FIG. 2 A-C, FIG. 2 A-C respectively show CDR1, CDR2, and CDR3 of variable regions of heavy chains (V_{H}) of rabbit anti-human PD-L1 mAbs in accordance with some embodiments including 2C5, 3B8, 8C6, 10H6, 11G7, and 12H2.

Referring to FIG. 2A, the V_{H} CDR1s of 2C5, 3B8, 8C6, 10H6, 11G7, and 12H2 can have a length of 9-10 amino acids. The amino acid sequence of the V_{H} CDR1 of 2C5 can include FSLNNDNVG (SEQ ID NO: 1). The amino acid sequence of the V_{H} CDR1 of 3B8 can include FSFSNNAYMC (SEQ ID NO: 2). The amino acid sequence of the V_{H} CDR1 of 8C6 can include FDISRHHIVIC (SEQ ID NO: 3). The amino acid sequence of the V_{H} CDR1 of 10H6 can include FSFSSIYYLC (SEQ ID NO: 4). The amino acid sequence of the V_{H} CDR1 of 11G7 can include IDLSSYDMT (SEQ ID NO: 5). The amino acid sequence of V_{H} CDR1 of 12H2 can include FSFSSSYYMC (SEQ ID NO: 6).

The V_{H} CDR1s of 3B8, 10H6, and 12H2 has a sequence formula of FSFS[S/N][S/I/N][Y/A]Y[M/L]C. The pair of square brackets “[ ]” represents a single position in a protein sequence, and the symbol “/” means “or”. For example, [A/B/C] matches any of the amino acids represented by A or B or C.

Referring to FIG. 2B, the V_{H} CDR2s of 2C5, 3B8, 8C6, 10H6, 11G7, and 12H2 have a length of 18-20 amino acids. The amino acid sequence of the V_{H} CDR2 of 2C5 can include WVGLIDILGSSYYASWAK (SEQ ID NO: 7). The amino acid sequence of the V_{H} CDR2 of 3B8 can include LIACIYSRYGNTHYASWAK (SEQ ID NO: 8). The amino acid sequence of the V_{H} CDR2 of 8C6 can include YIGFINPPGSAYYANWAK (SEQ ID NO: 9). The amino acid sequence of the V_{H} CDR2 of 10H6 can include WIACIYGGNSDNTWYASWAK (SEQ ID NO: 10). The amino acid sequence of the V_{H} CDR2 of 11G7 can include YIGYISYFDKTYYATWAK (SEQ ID NO: 11). The amino acid sequence of V_{H} CDR 2 of 12H2 can include WIACIWTSNGGRTYYANWAK (SEQ ID NO: 12).

Referring to FIG. 2C, the V_{H} CDR3s of 2C5, 3B8, 8C6, 10H6, 11G7, and 12H2 can have a length of 13-19 amino acids. The amino acid sequence of the V_{H} CDR3 of 2C5 can include YFCAREGDGSFDP (SEQ ID NO: 13). The amino acid sequence of the V_{H} CDR3 of 3B8 can include YFCARGASGSSDYSFYFNL (SEQ ID NO: 14). The amino acid sequence of the V_{H} CDR3 of 8C6 can include YFCARWRFDDYGDL (SEQ ID NO: 15). The amino acid sequence of the V_{H} CDR3 of 10H6 can include YFCARGYDIYGYAINL (SEQ ID NO: 16). The amino acid sequence of the V_{H} CDR3 of 11G7 can include YFCVRDRPDGITANL (SEQ ID NO: 17). The amino acid sequence of V_{H} CDR3 of 12H2 can include YFCARDDDTGSGYYNL (SEQ ID NO: 18).

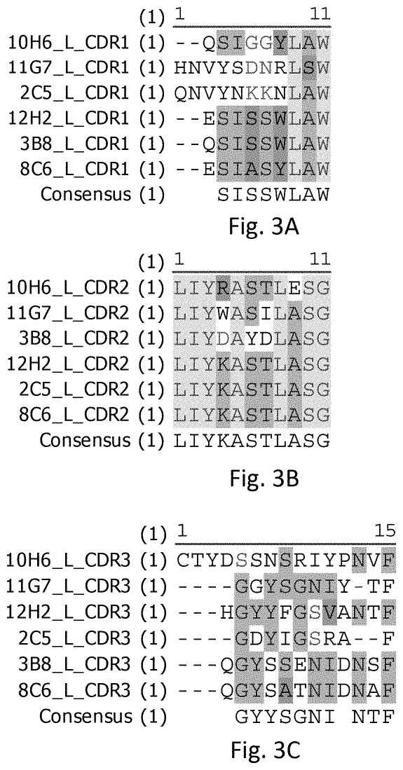

Referring to FIG. 3 A-C, FIG. 3 A-C respectively show CDR1, CDR2, and CDR3 of variable regions of Light chains (V_{L}) of rabbit anti-human PD-L1 mAbs in accordance with some embodiments including 2C5, 3B8, 8C6, 10H6, 11G7, and 12H2.

Referring to FIG. 3A, the V_{L} CDR1s of 2C5, 3B8, 8C6, 10H6, 11G7, and 12H2 can have a length of 9-11 amino acids. The amino acid sequence of the V_{L} CDR1 of 2C5 is QNVYNKKNLAW (SEQ ID NO: 19). The amino acid sequence of the V_{L} CDR1 of 3B8 is QSISSWLAW (SEQ ID NO: 20). The amino acid sequence of the V_{L} CDR1 of 8C6 is ESIASYLAW (SEQ ID NO: 21). The amino acid sequence of the V_{L} CDR1 of 10H6 is QSIGGYLAW (SEQ ID NO: 22). The amino acid sequence of the V_{L} CDR1 of 11G7 is HNVYSDNRLSW (SEQ ID NO: 23). The amino acid sequence of V_{L} CDR1 of 12H2 is ESISSWLAW (SEQ ID NO: 24). The V_{L} CDR1s of 2C5 and 11G7 can have a sequence formula of [H/Q]NVY[S/N][D/K][N/K][R/N]L[S/A]W. The V_{L} CDR1s of 3B8, 8C6, 10H6, and 12H2 can have a sequence formula of [Q/E]SI[S/G/A][S/G][Y/W]LAW.

Referring to FIG. 3B, the V_{L} CDR2s of 2C5, 3B8, 8C6, 10H6, 11G7, and 12H2 can have a length of 11 amino acids. The amino acid sequence of the V_{L} CDR2 of 2C5 can include LIYKASTLASG (SEQ ID NO: 25). The amino acid sequence of the V_{L} CDR2 of 3B8 can include LIYDAYDLASG (SEQ ID NO: 26). The amino acid sequence of the V_{L} CDR2 of 8C6 can include LIYKASTLASG (SEQ ID NO: 27). The amino acid sequence of the V_{L} CDR2 of 10H6 can include LIYRASTLESG (SEQ ID NO: 28). The amino acid sequence of the V_{L} CDR2 of 11G7 can include LIYWASILASG (SEQ ID NO: 29). The amino acid sequence of V_{L} CDR 2 of 12H2 can include LIYKASTLASG (SEQ ID NO: 30). The V_{L} CDR2s of 2C5, 3B8, 8C6, 10H6, 11G7, and 12H2 can have a sequence formula of LIY[K/R/W/D]A[S/Y][T/I/D]L[A/E]SG.

Referring to FIG. 3C, the V_{L} CDR3s of 2C5, 3B8, 8C6, 10H6, 11G7, and 12H2 can have a length of 9-15 amino acids. The amino acid sequence of the V_{L} CDR3 of 2C5 can include GDYIGSRAF (SEQ ID NO: 31). The amino acid sequence of the V_{L} CDR3 of 3B8 can include QGYSSENIDNS (SEQ ID NO: 32). The amino acid sequence of the V_{L} CDR3 of 8C6 can include QGYSATNIDNAF (SEQ ID NO: 33). The amino acid sequence of the V_{L} CDR3 of 10H6 can include CTYDSSNSRIYPNVF (SEQ ID NO: 34). The amino acid sequence of the V_{L} CDR3 of 11G7 can include GGYSGNIYTF (SEQ ID NO: 35). The amino acid sequence of V_{L} CDR3 of 12H2 can include HGYYFGSVANTF (SEQ ID NO: 36). The V_{L} CDR3s of 3B8 and 8C6 can have a sequence formula of QGYS[S/A][E/T]NIDN[S/A]F. The V_{L} CDR3s of 11G7 and 2C5 can have a sequence formula of G[G/D]Y[S/I]G[N/S][I/R][Y/A][T/-]F, where “-” indicates a deletion, i.e., an amino acid missing at the corresponding sequence position.

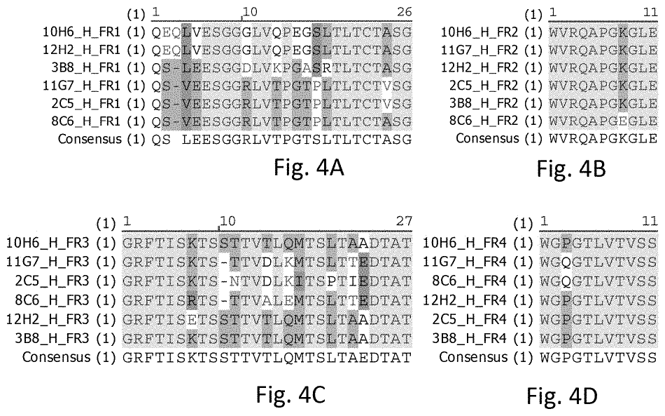

Referring to FIG. 4A-D, FIG. 4A-D respectively show V_{H} FR1, V_{H} FR2, V_{H} FR3, and V_{H} FR4 of rabbit anti-human PD-L1 mAbs in accordance with some embodiments including 2C5, 3B8, 8C6, 10H6, 11G7, and 12H2.

Referring to FIG. 4A, the V_{H} FR1 of 2C5, 3B8, 8C6, 10H6, 11G7, and 12H2 can have a length of 25-26 amino acids. The V_{H} FR1s of 2C5 and 11G7 each can include an amino acid sequence of QSVEESGGRLVTPGTPLTLTCTVSG (SEQ ID NO: 37). The V_{H} FR1 of 3B8 can include an amino acid sequence of QSLEESGGDLVKPGASRTLTCTASG (SEQ ID NO: 38). The V_{H} FR1 of 8C6 can include an amino acid sequence of QSVEESGGRLVTPGTPLTLTCTASG (SEQ ID NO: 39). The V_{H} FR1s of 10H6 and 12H2 can include an amino acid sequence of QEQLVESGGGLVQPEGSLTLTCTASG (SEQ ID NO: 40). The V_{H} FR1s of 2C5, 3B8, 8C6, 10H6, 11G7, and 12H2 can have a motif of Q[S/E][Q/-][L/V][E/V]ESGG[R/G/D]LV[T/Q/K]P[G/E][T/G/A][P/S][L/R]TLTCT[A/V]SG. The V_{H} FR1s of 2C5, 3B8, 8C6, 10H6, 11G7, and 12H2 can have a common structure of Qx[x/-]xxESGGxLVxPxxxxTLTCTxSG, where “x” represents a single amino acid site, and “[x/-]” represents a single amino acid site or a deletion. The V_{H} FR4s of 2C5, 8C6, 10H6, 11G7, and 12H2 can have 57.7% of identical amino acid residues and 96.2% of consensus amino acid residues.

Referring to FIG. 4B, the V_{H} FR2 of 2C5, 3B8, 8C6, 10H6, 11G7, and 12H2 can have a length of 11 amino acids. The V_{H} FR2s of 2C5, 3B8, 10H6, 11G7, and 12H2 each can include an amino acid sequence of WVRQAPGKGLE (SEQ ID NO: 41). The V_{H} FR2 of 8C6 can include an amino sequence of WVRQAPGEGLE (SEQ ID NO: 42). The V_{H} FR2s of 2C5, 3B8, 8C6, 10H6, 11G7, and 12H2 can have a common structure of WVRQAPGxGLE, where “x” represents a single amino acid site. The V_{H} FR2s of 2C5, 8C6, 10H6, 11G7, and 12H2 can have 90.9% of identical amino acid residues and 100% of consensus amino acid residues.

Referring to FIG. 4C, the V_{H} FR3 of 2C5, 3B8, 8C6, 10H6, 11G7, and 12H2 can have a length of 26-27 amino acids. The amino acid sequence of the V_{H} FR3 of 2C5 can include GRFTISKTSNTVDLKITSPTIEDTAT (SEQ ID NO: 43). The amino acid sequence of the V_{H} FR3 of 3B8 and 10 H6 can include GRFTISKTSSTTVTLQMTSLTAADTAT (SEQ ID NO: 44). The amino acid sequence of the V_{H} FR3 of 8C6 can include GRFTISRTSTTVALEMTSLTTEDTAT (SEQ ID NO: 45). The amino acid sequence of the V_{H} FR3 of 11G7 can include GRFTISKTSTTVDLKMTSLTTEDTAT (SEQ ID NO: 46). The amino acid sequence of V_{H} FR3 of 12H2 can include GRFTISETSSTTVTLQMTSLTAADTAT (SEQ ID NO: 47). The V_{H} FR3s of 2C5, 3B8, 8C6, 10H6, 11G7, and 12H2 can have a sequence motif or formula of GRFTIS[K/R/E]TS[SI-][T/N]TV[T/D/A]L[Q/K/E][M/I]TS[L/P]T[A/T/I][E/A]DTAT, where the “-” represents a deletion. The V_{H} FR3s of 2C5, 3B8, 8C6, 10H6, 11G7, and 12H2 can have a common structure of GRFTISxTS[x/-]xTVxLxxTSxTxxDTAT, where “x” represents a single amino acid site, and “[x/-]” represents a single amino acid site or a deletion. The V_{H} FR3s of 2C5, 8C6, 10H6, 11G7, and 12H2 can have 67.7% of identical amino acid residues and 100% of consensus amino acid residues.

Referring to FIG. 4C, the V_{H} FR4 of 2C5, 3B8, 8C6, 10H6, 11G7, and 12H2 can have a length of 11 amino acids. The V_{H} FR4s of 2C5, 3B8, 10H6, and 12H2 can have a common sequence or structure of WGPGTLVTVSS (SEQ ID NO: 48). The V_{H} FR4s of 8C6 and 11G7 can include a common sequence or structure of WGQGTLVTVSS (SEQ ID NO: 49). The V_{H} FR4s of 2C5, 3B8, 8C6, 10H6, 11G7, and 12H2 can have a motif or consensus sequence of WG[P/Q]GTLVTVSS. The V_{H} FR4s of 2C5, 3B8, 8C6, 10H6, 11G7, and 12H2 can have a common structure of WGxGTLVVTVSS, where “x” represents a single amino acid site. The V_{H} FR4s of 2C5, 8C6, 10H6, 11G7, and 12H2 can have 90.9% of identical amino acid residues and a 100% of consensus amino acid residues.

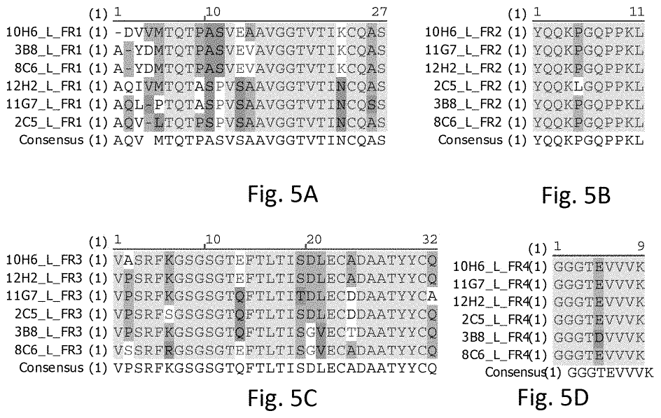

Referring to FIG. 5A-D, FIG. 4A-D respectively show FR1, FR2, FR3, and FR4 of light chains (V_{L}) of rabbit anti-human PD-L1 mAbs in accordance with some embodiments including 2C5, 3B8, 8C6, 10H6, 11G7, and 12H2.

Referring to FIG. 5A, the V_{L} FR1 of 2C5, 3B8, 8C6, 10H6, 11G7, and 12H2 have a length of 26-27 amino acids. The amino acid sequence of the V_{L} FR1 of 2C5 can include AQVLTQTPSPVSAAVGGTVTINCQAS (SEQ ID NO: 50). The amino acid sequence of the V_{L} FR1 of 3B8 and 8C6 can include AYDMTQTPASVEVAVGGTVTIKCQAS (SEQ ID NO: 51). The amino acid sequence of the V_{L} FR1 of 10H6 can include DVVMTQTPASVEAAVGGTVTIKCQAS (SEQ ID NO: 52). The amino acid sequence of the V_{L} FR1 of 11G7 can include AQLPTQTASPVSAAVGGTVTINCQSS (SEQ ID NO: 53). The amino acid sequence of V_{L} FR1 of 12H2 can include AQIVMTQTASPVSAAVGGTVTINCQAS (SEQ ID NO: 54). The V_{L} FR1s of 2C5, 3B8, 8C6, 10H6, 11G7, and 12H2 can have a sequence formula of [A/-][Q/D/-][Y/V/I/L][D/V/-][M/L/P]TQT[P/A][S/A][P/S]V[S/E][AN]AVGGTVTI[N/K]CQ[A/S]S. The V_{L} FR1s of 2C5, 3B8, 8C6, 10H6, 11G7, and 12H2 can have a common structure of TQTxxxVxxAVGGTVTIxCQxS, where “x” represents a single amino acid site. The V_{L} FR1s of 2C5, 8C6, 10H6, 11G7, and 12H2 can have 55.6% of identical amino acid residues and 96.3% of consensus amino acid residues.

Referring to FIG. 5B, the V_{L} FR2s of 2C5, 3B8, 8C6, 10H6, 11G7, and 12H2 can have a length of 11 amino acids. The amino acid sequence of the V_{L} FR2 of 2C5 can include YQQKLGQPPKL (SEQ ID NO: 55). The amino acid sequence of the V_{L} FR2 of 3B8, 8C6, 10H6, 11G7, and 12H2 can include YQQKPGQPPKL (SEQ ID NO: 56). The V_{L} FR2s of 2C5, 3B8, 8C6, 10H6, 11G7, and 12H2 can have a sequence formula of YQQK[P/L]GQPPKL. The V_{L} FR2s of 2C5, 3B8, 8C6, 10H6, 11G7, and 12H2 can have a common structure of YQQKxGQPPKL, where “x” represents a single amino acid. The V_{L} FR2s of 2C5, 8C6, 10H6, 11G7, and 12H2 can have 90.9% of identical amino acid residues and 100% of consensus amino acid residues.

Referring to FIG. 5C, the V_{L} FR3s of 2C5, 3B8, 8C6, 10H6, 11G7, and 12H2 can have a length of 32 amino acids. The amino acid sequence of the V_{L} FR3 of 2C5 can include VPSRFSGSGSGTQFTLTISDLECDDAATYYCQ (SEQ ID NO: 57). The amino acid sequence of the V_{L} FR3 of 3B8 can include VPSRFKGSGSGTQFTLTISGVECTDAATYYCQ (SEQ ID NO: 58). The amino acid sequence of the V_{L} FR3 of 8C6 can include VSSRFRGSGSGTEFTLTISGVECADAATYYCQ (SEQ ID NO: 59). The amino acid sequence of the V_{L} FR3 of 10H6 can include VASRFKGSGSGTEFTLTISDLECADAATYYCQ (SEQ ID NO: 60). The amino acid sequence of the V_{L} FR3 of 11G7 can include VPSRFKGSGSGTQFTLTITDLECDDAATYYCA (SEQ ID NO: 61). The amino acid sequence of V_{L} FR3 of 12H2 can include VPSRFKGSGSGTEFTLTISDLECADAATYYCQ (SEQ ID NO: 62). The V_{L} FR3 of 2C5, 3B8, 8C6, 10H6, 11G7, and 12H2 can have a sequence formula of V[P/A/S]SRF[K/S/R]GSGSGT[Q/E]FTLTI[S/T][D/G][L/V]EC[A/D/T]DAATYYC[Q/A]. The V_{L} FR3s of 2C5, 3B8, 8C6, 10H6, 11G7, and 12H2 can have a common structure of VxSRFx GSGSGTxFTLTIxxxECxDAATYCx, where “x” represents a single amino acid site. The V_{L} FR3s of 2C5, 8C6, 10H6, 11G7, and 12H2 can 75.0% of identical amino acid residues and 100% of consensus amino acid residues.

Referring to FIG. 5D, the V_{L} FR4 of 2C5, 3B8, 8C6, 10H6, 11G7, and 12H2 can have a length of 9 amino acids. The V_{L} FR4s of 2C5, 8C6, 10H6, 11G7, and 12H2 can include a common amino acid sequence of GGGTEVVVK (SEQ ID NO: 63). The V_{L} FR4 of 3B8 can include an amino acid sequence of GGGTDVVVK (SEQ ID NO: 64). The V_{L} FR4s of 2C5, 3B8, 8C6, 10H6, 11G7, and 12H2 have a sequence formula of GGGT[E/D]VVVK and a common structure of GGGTxVVVK. The V_{L} FR4s of 2C5, 8C6, 10H6, 11G7, and 12H2 can have 88.9% of identical amino acid residues and 100% of consensus amino acid residues.

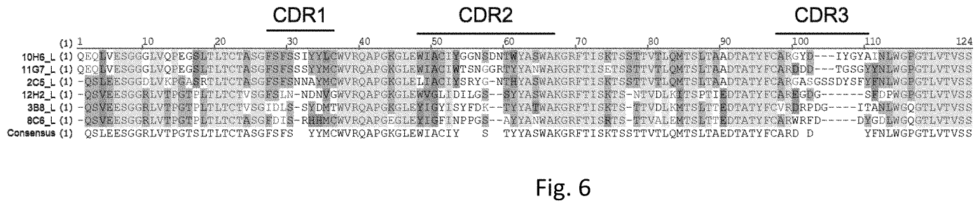

Referring to FIG. 6, FIG. 6 shows a sequence alignment of variable regions of heavy chains (V_{H}) of rabbit anti-human PD-L1 mAbs in accordance with some embodiments including 2C5, 3B8, 8C6, 10H6, 11G7, and 12H2.

The sequence of V_{H} of 2C5 can include QSVEESGGRLVTPGTPLTLTCTVSGFSLNNDNVGWVRQAPGKGLEWVGLIDILGSSYYA SWAKGRFTISKTSNTVDLKITSPTIEDTATYFCAREGDGSFDPWGPGTLVTVSS (SEQ ID NO: 65). The sequence of V_{H} of 3B8 can include QSLEESGGDLVKPGASRTLTCTASGFSFSNNAYMCWVRQAPGKGLELIACIYSRYGNTH YASWAKGRFTISKTSSTTVTLQMTSLTAADTATYFCARGASGSSDYSFYFNLWGPGTLV TVSS (SEQ ID NO: 66). The sequence of V_{H} of 8C6 can include QSVEESGGRLVTPGTPLTLTCTASGFDISRHHMCWVRQAPGEGLEYIGFINPPGSAYYAN WAKGRFTISRTSTTVALEMTSLTTEDTATYFCARWRFDDYGDLWGQGTLVTVSS (SEQ ID NO: 67). The sequence of V_{H} of 10H6 can include QEQLVESGGGLVQPEGSLTLTCTASGF SFS SIYYLCWVRQAPGKGLEWIACIYGGNSDN TWYASWAKGRFTISKTSSTTVTLQMTSLTAADTATYFCARGYDIYGYAINLWGPGTLVT VSS (SEQ ID NO: 68). The sequence of V_{H} of 11G7 can include QSVEESGGRLVTPGTPLTLTCTVSGIDLSSYDMTWVRQAPGKGLEYIGYISYFDKTYYAT WAKGRFTISKTSTTVDLKMTSLTTEDTATYFCVRDRPDGITANLWGQGTLVTVSS (SEQ ID NO: 69). The sequence of V_{H} of 12H2 can include QEQLVESGGGLVQPEGSLTLTCTASGFSFSSSYYMCWVRQAPGKGLEWIACIWTSNGGR TYYANWAKGRFTISETSSTTVTLQMTSLTAADTATYFCARDDDTGSGYYNLWGPGTLV TVSS (SEQ ID NO: 70). The V_{HS} of 2C5, 3B8, 8C6, 10H6, 11G7, and 12H2 can have 87.1% consensus amino acid residues and 50.0% of identical amino acid residues.

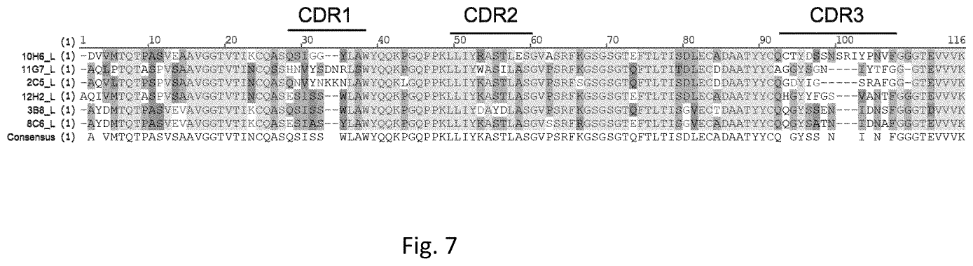

Referring to FIG. 7, FIG. 7 shows sequence alignment of variable regions of light chains (V_{L}) of rabbit anti-human PD-L1 mAbs in accordance with some embodiments including 2C5, 3B8, 8C6, 10H6, 11G7, and 12H2.

The sequence of V_{L} of 2C5 can include AQVLTQTPSPVSAAVGGTVTINCQASQNVYNKKNLAWYQQKLGQPPKWYKASTLAS GVPSRFSGSGSGTQFTLTISDLECDDAATYYCQGDYIGSRAFGGGTEVVVK (SEQ ID NO: 71). The sequence of V_{L} of 3B8 can include AYDMTQTPASVEVAVGGTVTIKCQASQSISSWLAWYQQKPGQPPKLLIYDAYDLASGV PSRFKGSGSGTQFTLTISGVECTDAATYYCQQGYSSENIDNSFGGGTDVVVK (SEQ ID NO: 72). The sequence of V_{L} of 8C6 can include AYDMTQTPASVEVAVGGTVTIKCQASESIASYLAWYQQKPGQPPKLLIYKASTLASGVS SRFRGSGSGTEFTLTISGVECADAATYYCQQGYSATNIDNAFGGGTEVVVK (SEQ ID NO: 73). The sequence of V_{L} of 10H6 can include DVVMTQTPASVEAAVGGTVTIKCQASQSIGGYLAWYQQKPGQPPKLLIYRASTLESGVA SRFKGSGSGTEFTLTISDLECADAATYYCQCTYDSSNSRIYPNVFGGGTEVVVK (SEQ ID NO: 74). The sequence of V_{L} of 11G7 can include AQLPTQTASPVSAAVGGTVTINCQSSHNVYSDNRLSWYQQKPGQPPKLLIYWASILASG VPSRFKGSGSGTQFTLTITDLECDDAATYYCAGGYSGNIYTFGGGTEVVVK (SEQ ID NO: 75). The sequence of V_{L} of 12H2 can include AQIVMTQTASPVSAAVGGTVTINCQASESIS SWLAWYQQKPGQPPKLLIYKASTLASGV PSRFKGSGSGTEFTLTISDLECADAATYYCQHGYYFGSVANTFGGGTEVVVK (SEQ ID NO: 76). The V_{L}s of 2C5, 3B8, 8C6, 10H6, 11G7, and 12H2 can have 90.5% consensus amino acid residues and 56.9% of identical amino acid residues.

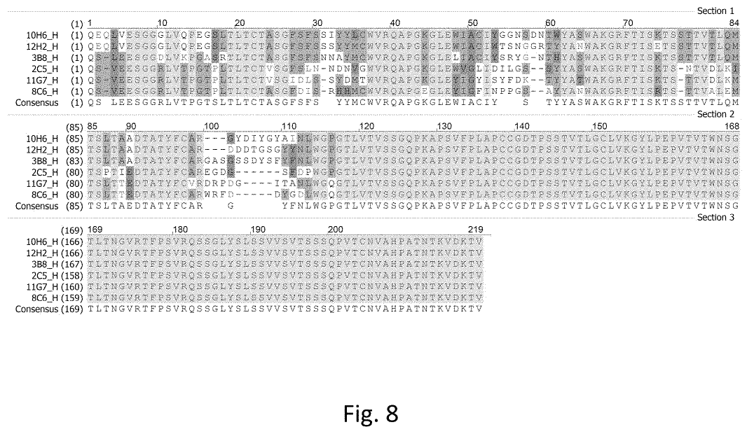

Referring to FIG. 8, FIG. 8 shows a sequence alignment of heavy chains of Fab fragments of the rabbit anti-human PD-L1 mAbs in accordance with some embodiments including 2C5, 3B8, 8C6, 10H6, 11G7, and 12H2.

The sequence of the heavy chain of 2C5 Fab fragment can include QSVEESGGRLVTPGTPLTLTCTVSGFSLNNDNVGWVRQAPGKGLEWVGLIDILGSSYYA SWAKGRFTISKTSNTVDLKITSPTIEDTATYFCAREGDGSFDPWGPGTLVTVS SGQPKAP SVFPLAPCCGDTPSSTVTLGCLVKGYLPEPVTVTWNSGTLTNGVRTFPSVRQSSGLYSLS SVVSVTSSSQPVTCNVAHPATNTKVDKTV (SEQ ID NO: 77). The sequence of the heavy chain of 3B8 Fab fragment can include QSLEESGGDLVKPGASRTLTCTASGFSFSNNAYMCWVRQAPGKGLELIACIYSRYGNTH YASWAKGRFTISKTSSTTVTLQMTSLTAADTATYFCARGASGSSDYSFYFNLWGPGTLV TVSSGQPKAPSVFPLAPCCGDTPSSTVTLGCLVKGYLPEPVTVTWNSGTLTNGVRTFPSV RQSSGLYSLSSVVSVTSSSQPVTCNVAHPATNTKVDKTV (SEQ ID NO: 78). The sequence of the heavy chain of 8C6 Fab fragment can include QSVEESGGRLVTPGTPLTLTCTASGFDISRHHMCWVRQAPGEGLEYIGFINPPGSAYYAN WAKGRFTISRTSTTVALEMTSLTTEDTATYFCARWRFDDYGDLWGQGTLVTVSSGQPK APSVFPLAPCCGDTPSSTVTLGCLVKGYLPEPVTVTWNSGTLTNGVRTFPSVRQSSGLYS LSSVVSVTSSSQPVTCNVAHPATNTKVDKTV (SEQ ID NO: 79). The sequence of the heavy chain of 10H6 Fab fragment can include QEQLVESGGGLVQPEGSLTLTCTASGF SFS SIYYLCWVRQAPGKGLEWIACIYGGNSDN TWYASWAKGRFTISKTSSTTVTLQMTSLTAADTATYFCARGYDIYGYAINLWGPGTLVT VSSGQPKAPSVFPLAPCCGDTPSSTVTLGCLVKGYLPEPVTVTWNSGTLTNGVRTFPSVR QSSGLYSLSSVVSVTSSSQPVTCNVAHPATNTKVDKTV (SEQ ID NO: 80). The sequence of the heavy chain of 11G7 Fab fragment can include QSVEESGGRLVTPGTPLTLTCTVSGIDLSSYDMTWVRQAPGKGLEYIGYISYFDKTYYAT WAKGRFTISKTSTTVDLKMTSLTTEDTATYFCVRDRPDGITANLWGQGTLVTVSSGQPK APSVFPLAPCCGDTPSSTVTLGCLVKGYLPEPVTVTWNSGTLTNGVRTFPSVRQSSGLYS LSSVVSVTSSSQPVTCNVAHPATNTKVDKTV (SEQ ID NO: 81). The sequence of the heavy chain of 12H2 Fab fragment can include QEQLVESGGGLVQPEGSLTLTCTASGFSFSSSYYMCWVRQAPGKGLEWIACIWTSNGGR TYYANWAKGRFTISETSSTTVTLQMTSLTAADTATYFCARDDDTGSGYYNLWGPGTLV TVSSGQPKAPSVFPLAPCCGDTPSSTVTLGCLVKGYLPEPVTVTWNSGTLTNGVRTFPSV RQSSGLYSLSSVVSVTSSSQPVTCNVAHPATNTKVDKTV (SEQ ID NO: 82). The heavy chains of Fab fragments of 2C5, 3B8, 8C6, 10H6, 11G7, and 12H2 can have 92.2% consensus amino acid residues and 71.7% of identical amino acid residues.

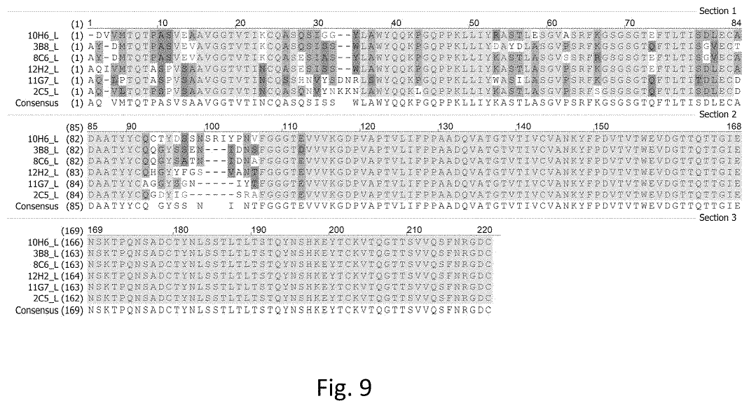

Referring to FIG. 9, FIG. 9 shows a sequence alignment of light chains of Fab fragments of rabbit anti-human PD-L1 mAbs in accordance with some embodiments including 2C5, 3B8, 8C6, 10H6, 11G7, and 12H2.

The sequence of the light chain of 2C5 Fab fragment can include AQVLTQTPSPVSAAVGGTVTINCQASQNVYNKKNLAWYQQKLGQPPKWYKASTLAS GVPSRFSGSGSGTQFTLTISDLECDDAATYYCQGDYIGSRAFGGGTEVVVKGDPVAPTV LIFPPAADQVATGTVTIVCVANKYFPDVTVTWEVDGTTQTTGIENSKTPQNSADCTYNL SSTLTLTSTQYNSHKEYTCKVTQGTTSVVQSFNRGDC (SEQ ID NO: 83). The sequence of the light chain of 3B8 Fab fragment can include AYDMTQTPASVEVAVGGTVTIKCQASQSISSWLAWYQQKPGQPPKLLIYDAYDLASGV PSRFKGSGSGTQFTLTISGVECTDAATYYCQQGYSSENIDNSFGGGTDVVVKGDPVAPT VLIFPPAADQVATGTVTIVCVANKYFPDVTVTWEVDGTTQTTGIENSKTPQNSADCTYN LSSTLTLTSTQYNSHKEYTCKVTQGTTSVVQSFNRGDC (SEQ ID NO: 84). The sequence of the light chain of 8C6 Fab fragment can include AYDMTQTPASVEVAVGGTVTIKCQASESIASYLAWYQQKPGQPPKLLIYKASTLASGVS SRFRGSGSGTEFTLTISGVECADAATYYCQQGYSATNIDNAFGGGTEVVVKGDPVAPTV LIFPPAADQVATGTVTIVCVANKYFPDVTVTWEVDGTTQTTGIENSKTPQNSADCTYNL SSTLTLTSTQYNSHKEYTCKVTQGTTSVVQSFNRGDC (SEQ ID NO: 85). The sequence of the light chain of 10H6 Fab fragment can include DVVMTQTPASVEAAVGGTVTIKCQASQSIGGYLAWYQQKPGQPPKLLIYRASTLESGVA SRFKGSGSGTEFTLTISDLECADAATYYCQCTYDSSNSRIYPNVFGGGTEVVVKGDPVAP TVLIFPPAADQVATGTVTIVCVANKYFPDVTVTWEVDGTTQTTGIENSKTPQNSADCTY NLSSTLTLTSTQYNSHKEYTCKVTQGTTSVVQSFNRGDC (SEQ ID NO: 86). The sequence of the light chain of 11G7 Fab fragment can include AQLPTQTASPVSAAVGGTVTINCQSSHNVYSDNRLSWYQQKPGQPPKLLIYWASILASG VPSRFKGSGSGTQFTLTITDLECDDAATYYCAGGYSGNIYTFGGGTEVVVKGDPVAPTV LIFPPAADQVATGTVTIVCVANKYFPDVTVTWEVDGTTQTTGIENSKTPQNSADCTYNL SSTLTLTSTQYNSHKEYTCKVTQGTTSVVQSFNRGDC (SEQ ID NO: 87). The sequence of the light chain of 12H2 Fab fragment can include AQIVMTQTASPVSAAVGGTVTINCQASESIS SWLAWYQQKPGQPPKLLIYKASTLASGV PSRFKGSGSGTEFTLTISDLECADAATYYCQHGYYFGSVANTFGGGTEVVVKGDPVAPT VLIFPPAADQVATGTVTIVCVANKYFPDVTVTWEVDGTTQTTGIENSKTPQNSADCTYN LSSTLTLTSTQYNSHKEYTCKVTQGTTSVVQSFNRGDC (SEQ ID NO: 88). The light chains of Fab fragments of 2C5, 3B8, 8C6, 10H6, 11G7, and 12H2 can have 95.9% consensus amino acid residues and 78.2% of identical amino acid residues.

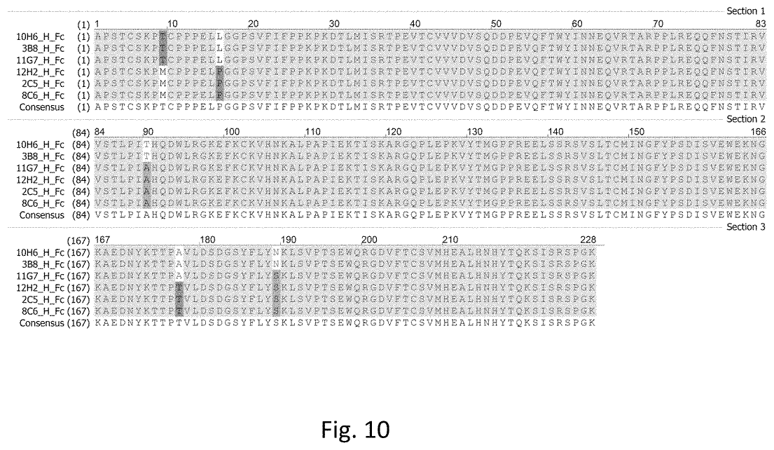

Referring to FIG. 10, FIG. 10 shows a sequence alignment of heavy chains of Fc fragments of rabbit anti-human PD-L1 mAbs in accordance with some embodiments including 2C5, 3B8, 8C6, 10H6, 11G7, and 12H2.

The sequence of the heavy chains of Fc fragments of 2C5, 8C6, and 12H2 can include APSTCSKPMCPPPELPGGPSVFIFPPKPKDTLMISRTPEVTCVVVDVSQDDPEVQFTWYIN NEQVRTARPPLREQQFNSTIRVVSTLPIAHQDWLRGKEFKCKVHNKALPAPIEKTISKAR GQPLEPKVYTMGPPREELSSRSVSLTCMINGFYPSDISVEWEKNGKAEDNYKTTPTVLDS DGSYFLYSKLSVPTSEWQRGDVFTCSVMHEALHNHYTQKSISRSPGK (SEQ ID NO: 89). The sequence of the heavy chain of Fc fragments of 3B8 and 10H6 can include APSTCSKPTCPPPELLGGPSVFIFPPKPKDTLMISRTPEVTCVVVDVSQDDPEVQFTWYIN NEQVRTARPPLREQQFNSTIRVVSTLPITHQDWLRGKEFKCKVHNKALPAPIEKTISKAR GQPLEPKVYTMGPPREELSSRSVSLTCMINGFYPSDISVEWEKNGKAEDNYKTTPAVLD SDGSYFLYNKLSVPTSEWQRGDVFTCSVMHEALHNHYTQKSISRSPGK (SEQ ID NO: 90). The sequence of the heavy chain of Fc fragment of 11G7 can include APSTCSKPTCPPPELLGGPSVFIFPPKPKDTLMISRTPEVTCVVVDVSQDDPEVQFTWYIN NEQVRTARPPLREQQFNSTIRVVSTLPIAHQDWLRGKEFKCKVHNKALPAPIEKTISKAR GQPLEPKVYTMGPPREELSSRSVSLTCMINGFYPSDISVEWEKNGKAEDNYKTTPAVLD SDGSYFLYSKLSVPTSEWQRGDVFTCSVMHEALHNHYTQKSISRSPGK (SEQ ID NO: 91). The heavy chains of Fc fragments of 2C5, 3B8, 8C6, 10H6, 11G7, and 12H2 can have 100% consensus amino acid residues and 97.8% of identical amino acid residues.

In an embodiment, the rabbit anti-PD-L1 mAb can be an antigen-binding portion of the Y-shaped antibodies disclosed herein. In an embodiment, the rabbit anti-PD-L1 mAb can be a Fab fragment that is a monovalent fragment formed of the V_{L}, V_{H}, C_{L} and C_{H1} domains. In an embodiment, the rabbit anti-PD-L1 mAb can be an F(ab′)₂ fragment that is a bivalent fragment including the two Fab fragments 7 linked by, e.g., a disulfide bridge of the hinge region 10. In an embodiment, the rabbit anti-PD-L1 mAb can be an Fd fragment formed of the V_{H} and C_{H1} domains. In an embodiment, the rabbit anti-PD-L1 mAb can be the Fv fragment 9 formed of the V_{L} and V_{H} domains. In an embodiment, the rabbit anti-PD-L1 mAb can be a single-domain antibody (sdAb) composed of a V_{H} domain of a full-length antibody. In an embodiment, the rabbit anti-PD-L1 mAb can be an isolated complementarity-determining region.

The rabbit anti-PD-L1 mAb can also encompass structures derived from the embodiments or their antigen-binding portions by genetic modification. Different genetically modified antibody structures can be generated, including but not being limited to humanized antibodies, chimeric antibodies, etc. In an embodiment, the rabbit anti-PD-L1 mAb can be a humanized antibody, as its protein sequence has been modified to increase its similarity to antibody variants produced naturally in humans. The protein sequence of a humanized antibody can be essentially identical to that of a human variant, despite the rabbit origin of some of its CDRs that are essential to the ability of the antibody to bind to human PD-L1. In an embodiment, a humanized antibody can be created by insertion of CDRs of a non-human antibody, e.g., a rabbit antibody, into a human antibody scaffold. In an embodiment, the rabbit anti-PD-L1 mAb can be a chimeric antibody. In an embodiment, the chimeric antibody can be an antibody made by fusing variable regions of the heavy and light chains from the Y-shaped antibodies herein, with the constant regions from another species such as a human. In an embodiment, the chimeric antibody is an antibody made by fusing Fab of one of the rabbit anti-PD-L1 mAbs disclosed herein with Fc of a human antibody. In an embodiment, the rabbit anti-PD-L1 mAb can be a single-chain Fv (scFv). Although the two domains of the Fv fragment, namely V_{L} and V_{H}, are coded by separate genes, they can be joined, using recombinant methods, by a synthetic linker to form the scFv. In an embodiment, the genetic modification can be performed in accordance with methods known in the art, and the genetically modified antibody structures can be screened for utility in the same manner as the full-length antibodies.

The antibody disclosed herein can also encompass structures derived from the embodiments disclosed herein and their antigen-binding portions by chemical modification. In an embodiment, the chemical modification can be a chemical crosslinking. In an embodiment, one or more conjugates can be covalently linked to or non-covalently attached to the antibody. In an embodiment, the conjugate can be a molecular label covalently attached to the antibody to facilitate the detection of its antigen. The conjugates can be a small molecule. In an embodiment, the small molecule can be biotin. In an embodiment, the small molecule can be streptavidin. In an embodiment, the small molecule can be a fluorescent dye. The fluorescent dye includes but not limited to Alexa fluors, aminomethylcoumarin (AMCA), Atto dyes, cyanine dyes, DyLight fluors, FITC, FluoProbes 647H, Rhodamine, and Texas Red. The Alexa fluors include but not limited to Alexa Fluor 488, Alexa Fluor 555, Alexa Fluor 568, Alexa Fluor 594, Alexa Fluor 647, and Alexa Fluor 700. The Atto dyes include but not limited to Atto 390, Atto 488, Atto 565, Atto 633, and Atto 700. The cyanine dyes include but not limited to Cy3, Cy5, and Cy5.5. The DyLight dyes include but not limited to DyLight 350, DyLight 405, DyLight 488, DyLight 550, DyLight 594, DyLight 633, DyLight 650, DyLight 680, DyLight 755, and DyLight 800. In an embodiment, the conjugates can be a tandem dye that can have two covalently attached fluorescence molecules. In an embodiment, one of the fluorescence molecules serves as a donor, and the other serves as an acceptor. In an embodiment, the donor and the acceptor can behave as a unique fluorophore with the excitation properties of the donor and the emission properties of the acceptor. The tandem dye can include but not limited to Allophycocyanin-Cy5.5, Allophycocyanin-Cy 7, PE-Atto 594, PE-Cy 5, PE-Cy 5.5, PE-Cy 7, PE-Texas Red, PE-Alexa Fluor 647, PE-Alexa Fluor 700, PE-Alexa Fluor 750, APC-Alexa Fluor 750, and PerCP-Cy5.5.

The conjugates can also be a large molecule. In an embodiment, the large molecule can be an enzyme. The enzyme can include but not limited to alkaline phosphatase (AP), glucose oxidase (Gox), Horseradish peroxidase (HRP). In an embodiment, the large molecule can be a fluorescent protein. The fluorescent protein can include but not limited to Allophycocyanin (APC), B-Phycoerythrin (BPE), R-Phycoerythrin (R-PE), PerCP, and R-Phycocyanin (RPC). In an embodiment, the large molecule can also be an antibody whose specificity differs from that of the rabbit monoclonal anti-human PD-L1 antibody, forming a tandem antibody with multiple specificities.

Anti-human PD-L1 antibodies can have various in vivo and in vitro applications including but not limited to immunoassays, immunostaining, immunohistochemistry, diagnosis of human conditions associated with PD-L1, immuno-oncology therapies, and treatment of some infectious diseases caused by PD-L1. The immunoassays can include enzyme-linked immunosorbent assay (ELISA). The rabbit anti-human PD-L1 antibodies can be used in different forms of ELISA.

In an embodiment, the anti-human PD-L1 antibodies disclosed can be used in a direct ELISA. A direct ELISA can be a plate-based immunosorbent assay intended for the detection and quantification of a specific antigen from or within a complex biological sample.

There are varieties of methods for performing direct ELISA. In an embodiment, the antigen, e.g., extracellular domain of human PD-L1, can be immobilized or adsorbed onto a surface of a plastic plate. In an embodiment, the plastic plate can be a multi-well microtiter plate. In an embodiment, the multi-well microtiter plane can be a 96-well polystyrene plate. In an embodiment, an excessive amount of blocking protein can be added onto the surface to block all the other binding sites. In an embodiment, the blocking protein is bovine serum albumin. In an embodiment, an antibody specific for the antigen, e.g., extracellular domain of human PD-L1, can be added onto the surface to form a complex with the antigen. In an embodiment, the antibody can be conjugated with an enzyme. In an embodiment, the enzyme can be HRP. After the excess conjugated antibody is washed off, the conjugated antibody bound to the antigen stay. In an embodiment, by adding a substrate, the conjugated antibody can catalyze a reaction with the substrate, resulting in a visible colorimetric output that can be measured by a spectrophotometer or absorbance microplate reader. Direct ELISA, when compared to other forms of ELISA testing, can be performed faster because only one antibody is used and fewer steps are required. In an embodiment, the direct ELISA can test specific antibody-to-antigen reactions and helps to eliminate cross-reactivity between other antibodies. Direct ELISA is suitable for qualitative and quantitative antigen detection in samples of interest, antibody screening, and epitope mapping.

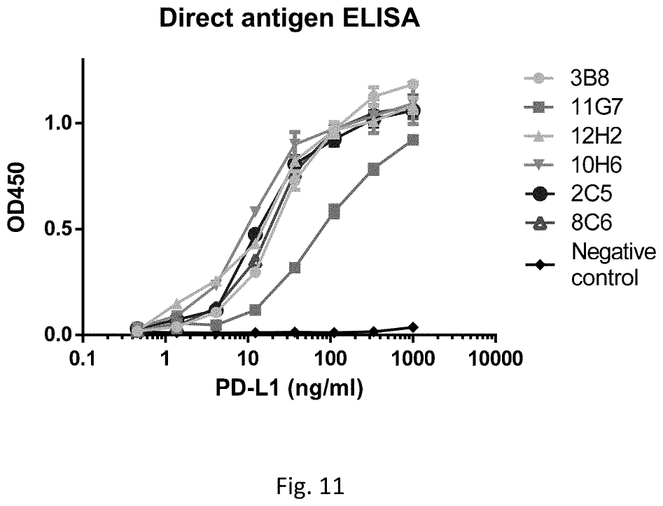

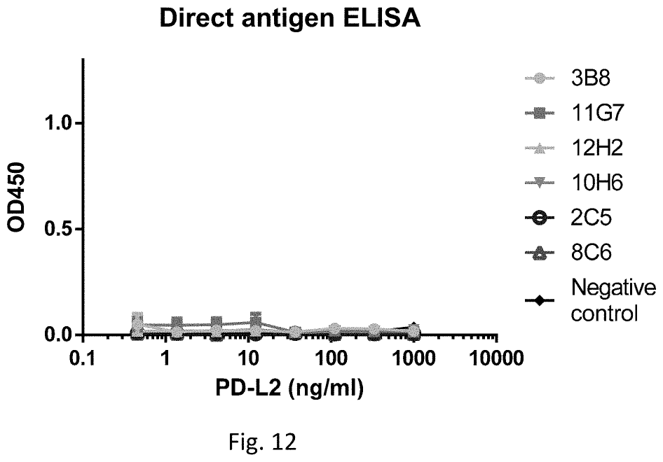

FIG. 11 shows standard curves of 2C5, 3B8, 8C6, 10H6, 11G7, and 12H2 in a direct ELISA for human PD-L1 in accordance with an embodiment. The procedures of the direct ELISA are described in the method below. Referring to FIG. 12, the X-axis shows the concentrations of the test sample human PD-L1 with a unit of ng/ml, and the Y-axis shows the optical density at the wavelength of 450 nm for the concentrations up to 1000 ng/ml. As seen, all the anti-human PD-L1 antibodies exhibit excellent “S” curves. The negative control was conducted using the same procedures as the anti-human PD-L1 antibodies, except that a blank buffer is used instead of the anti-human PD-L1 antibodies. The blank buffer is the buffer for diluting the anti-human PD-L1 antibodies. As compared to the curve for the negative control, 2C5, 3B8, 8C6, 10H6, 11G7, and 12H2 exhibit excellent sensitivity to a wide range of concentrations of human PD-L1.

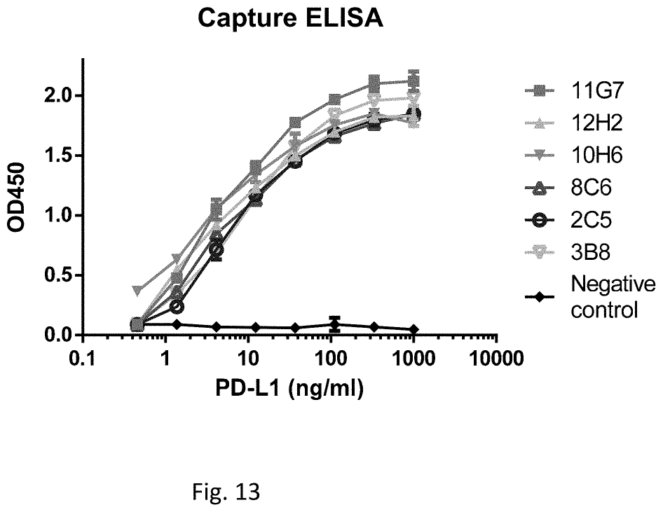

FIG. 12 shows standard curves of 2C5, 3B8, 8C6, 10H6, 11G7, and 12H2 in a direct ELISA for human PD-L2 in accordance with an embodiment. The procedures of the direct ELISA are described in the method below. Referring to FIG. 13, the X-axis shows the concentrations of the test sample human PD-L2 with a unit of ng/ml, and the Y-axis shows the optical density at the wavelength of 450 nm for the concentrations up to 1000 ng/ml. The negative control was conducted using the same procedures as the anti-human PD-L1 antibodies, except that a blank buffer is used instead of the anti-human PD-L1 antibodies. The blank buffer is the buffer for diluting the anti-human PD-L1 antibodies. As compared to the curve of the negative control, 2C5, 3B8, 8C6, 10H6, 11G7, and 12H2 does not recognize and bind human PD-L2. Thereby, 2C5, 3B8, 8C6, 10H6, 11G7, and 12H2 can eliminate the cross-reactivity of human PD-L2.

In an embodiment, the anti-human PD-L1 antibodies disclosed can be used in a capture ELISA. The capture ELISA is an alternative direct ELISA, where a monoclonal antibody is used to attach the antigen, e.g., human PD-L1, to the plastic plate. As compared to the direct ELISA, the capture ELISA has the advantage of preventing the surface of the plastic plate from blocking or denaturing the antigen. In an embodiment, the capture ELISA can give a higher sensitivity than that of the direct ELISA, as epitopes on human PD-L1 can be more efficiently exposed. In an embodiment, the capture ELISA can also be used in screening and testing an antibody capable of recognizing an epitope in a native conformation.

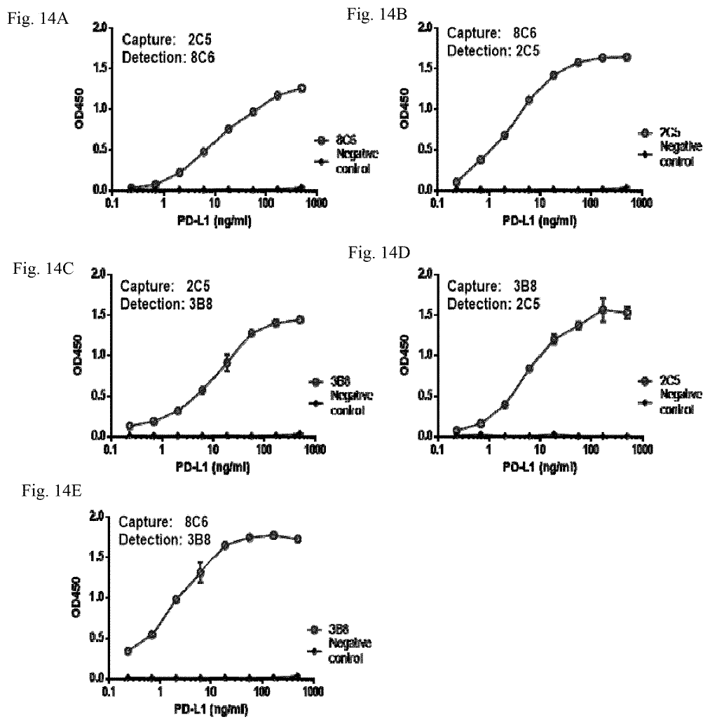

FIG. 13 shows standard curves of 2C5, 3B8, 8C6, 10H6, 11G7, and 12H2 in a capture ELISA for human PD-L1 in accordance with an embodiment. The procedures of the capture ELISA are described in the method below. Referring to FIG. 14, the X-axis shows concentrations of the test sample human PD-L1 with a unit of ng/ml, and the Y-axis shows the optical density at the wavelength of 450 nm for the concentrations up to 1000 ng/ml. As compared to the direct ELISA in FIG. 11, the capture ELISA can generally exhibit two times higher sensitivity. The capture ELISA gives about two times higher OD₄₅₀ values than those of the direct ELISA. For example, the OD₄₅₀ value for 2C5 is slightly higher than 1.0 in the capture ELISA at 10 ng/ml. In contrast, the OD₄₅₀ value 2C5 is slightly lower than 0.5 in the direct ELISA at 10 ng/ml of human PD-L1 (see FIG. 12). The capture ELISA can even improve the sensitivity of 11G7 to 15 times higher than that of the direct ELISA. For example, the OD₄₅₀ value for 11G7 is about 0.1 in the direct ELISA at 10 ng/ml of human PD-L1 (see FIG. 12), but the OD₄₅₀ value for 11G7 is close to 1.5 in capture ELISA at 10 ng/ml.

As also seen in FIG. 14, all the anti-human PD-L1 antibodies disclosed herein exhibit excellent “S” curves. The negative control was conducted using the same procedures as the anti-human PD-L1 antibodies, except that a blank buffer is used instead of the anti-human PD-L1 antibodies. The blank buffer is the buffer for diluting the anti-human PD-L1 antibodies. As compared to the curve for the negative control, 2C5, 3B8, 8C6, 10H6, 11G7, and 12H2 exhibit excellent sensitivity to a wide range of concentrations of human PD-L1.

In an embodiment, the anti-human PD-L1 antibodies disclosed can be used in a sandwich ELISA. Sandwich ELISA can be efficient in sample antigen detection. A sandwich ELISA detects and/or quantifies an antigen between two layers of antibody pairs: a capture and a detection antibody. The antigen to be measured contains at least two antigenic epitopes capable of binding to the pair of antibodies without interference. The sandwich ELISA can be very sensitive and does not require the antigen to be purified before analysis. In an embodiment, the sandwich ELISA can be up to at least two times more sensitive to human PD-L1 than the direct ELISA.

There are various methods or steps for performing the sandwich ELISA. In an embodiment, the capture antibody is immobilized or coated on a surface of a plastic plate. In an embodiment, a test sample, e.g., human PD-L1, containing a first antigenic epitope and a second antigenic epitope can be added to the surface. In an embodiment, the capture antibody binds the first antigenic epitope. In an embodiment, the detecting antibody can be added and binds to the second antigenic epitope. In an embodiment, an enzyme-linked secondary antibody can be added and binds to the detecting antibody. In an embodiment, a substrate can be added, and the enzyme linked to the secondary antibody catalyzes a reaction with the substrate, resulting in a visible colorimetric output that can be measured by a spectrophotometer or absorbance microplate reader.

FIGS. 14 A-E show results of sandwich ELISA using the rabbit anti-human PD-L1 mAbs as capture and detection antibodies, in accordance with an embodiment. The procedures of the capture ELISA are described in the method below. The X-axis shows concentrations of the test sample human PD-L1 with a unit of ng/ml, and the Y-axis shows OD₄₅₀ (the optical density at the wavelength of 450 nm) at the concentrations of human PD-L1. The negative control was conducted using the same procedures as the anti-human PD-L1 antibodies, except that a blank buffer is used instead of the anti-human PD-L1 antibodies. The blank buffer is the buffer for diluting the anti-human PD-L1 antibodies.

Referring to FIG. 14A, FIG. 14A shows results of the sandwich ELISA using the rabbit anti-human PD-L1 mAbs 2C5 as a capture antibody and 8C6 as a detection antibody. The negative control exhibits no significant OD₄₅₀ value. However, using 8C6 as a detective antibody gives higher and higher OD₄₅₀ value as the concentration of human PD-L1 increases, resulting in a curve with an excellent “S” shape. The results demonstrate that the binding of the capture antibody 2C5 to human PD-L1 does not prevent the detective antibody 8C6 from binding the human PD-L1. The results also show that 2C5 and 8C6 bind to different antigenic epitopes of human PD-L1.

As compared to the direct ELISA with 8C6 shown in FIG. 11, the sandwich ELISA with 8C6 as a detective antibody gives substantially higher OD₄₅₀ value at a concentration of human PD-L1. The sandwich ELISA enhances the sensitivity of human PD-L1 to 2C5, as compared to the direct ELISA.

Referring to FIG. 14B, FIG. 14B shows results of sandwich ELISA using the rabbit anti-human PD-L1 mAbs 8C6 as the capture antibody and 2C5 as the detection antibody. As seen, 2C5 as a detective antibody gives higher and higher OD₄₅₀ value as the concentration of human PD-L1 increases, resulting in a curve with an excellent “s” shape. In contrast, the negative control exhibits no significant OD₄₅₀ value as the concentration of human PD-L1 increases. The results demonstrate that the binding of the capture antibody 8C6 to human PD-L1 does not prevent the detective antibody 2C5 from binding the human PD-L1. The results also show that 8C6 and 2C5 bind to different antigenic epitopes on human PD-L1.

As compared to the direct ELISA with 2C5 shown in FIG. 11, the sandwich ELISA with 2C5 as a detective antibody gives an OD₄₅₀ value higher than 1.0 at 10 ng/ml of human PD-L1. However, the direct ELISA with 2C5 gives an OD₄₅₀ value of about 0.5 at 10 ng/ml of human PD-L1. The sandwich ELISA with 2C5 as a detective antibody is at least two times more sensitive to human PD-L1 than the direct ELISA.

FIGS. 14A and B demonstrate that 2C5 and 8C6 can be a pair of antibodies used in sandwich ELISA.

FIG. 14C shows results of sandwich ELISA using the rabbit anti-human PD-L1 mAbs 2C5 as the capture antibody and 3B8 as the detection antibody. As seen, the binding of 2C5 to human PD-L1 does not interfere with the ability of 3B8 to bind the human PD-L1.

FIG. 14D shows results of sandwich ELISA using the rabbit anti-human PD-L1 mAbs 3B8 as the capture antibody and 2C5 as the detection antibody. As seen, the binding of 3B8 to human PD-L1 does not prevent 2C5 from binding to the human PD-L1.

FIGS. 14C and D demonstrate that 2C5 and 3B8 can be a pair of antibodies used in sandwich ELISA. 2C5 and 3B8 bind to the different antigenic epitopes on human PD-L1.

FIG. 14E shows results of sandwich ELISA using the rabbit anti-human PD-L1 mAbs 8C6 as the capture antibody and 3B8 as a detective antibody. As seen, the binding of 8C6 to human PD-L1 does not interfere with the ability of 3B8 to bind the human PD-L1. FIG. 14E at least demonstrates that 8C6 and 3B8 can be used as capture and detective antibodies in a sandwich ELISA, respectively. 8C6 and 3B8 bind to the different antigenic epitopes at different locations of the extracellular domain of human PD-L1.

The ELISA including those discussed above can be performed with ELISA kits for the detection and quantification of human PD-L1. In an embodiment, the ELISA kit can include an anti-human PD-L1 mAb as a detection antibody. In an embodiment, the anti-human PD-L1 mAb is a rabbit anti-human PD-L1 mAb. In an embodiment, the rabbit anti-human PD-L1 mAb is 2C5, 3B8, 8C6, 10H6, 11G7, or 12H2, or an antigen-binding portion thereof. In an embodiment, the rabbit anti-human PD-L1 mAb can include a derivative and/or conservative modification. In an embodiment, the ELISA kit can further include an ELISA plate. In an embodiment, the ELISA kit can further include a second antibody. In an embodiment, the second antibody is a conjugated antibody.

Given that each of 2C5, 3B8, 8C6, 10H6, 11G7, and 12H2 can bind to PD-L1 and that antigen-binding specificity is provided primarily by the CDRs, the CDR sequences from different antibodies can be mixed and matched to create other anti-human PD-L1 antibodies. When V_{H} CDR sequences are mixed and matched, the CDR1, CDR2 and/or CDR3 sequence from a particular Vu sequence is preferably replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It is readily apparent to the ordinarily skilled artisan that novel Vu and V_{L} sequences can be created by substituting one or more Vu and/or V_{L} CDR region sequences with structurally similar sequences from the CDR sequences disclosed.

The V_{H}, V_{L}, light chain, and heavy chain sequences (amino acid sequences and the nucleotide sequences encoding the amino acid sequences) can also be “mixed and matched” to create other PD-L1-binding antibodies. When Vu and V_{L} sequences are mixed and matched, a Vu sequence from a particular V_{H}/V_{L} pairing is preferably replaced with a structurally similar Vu sequence, and a V_{L} sequence from a particular V_{H}/V_{L} pairing is preferably replaced with a structurally similar V_{L} sequence. Likewise, when heavy chain and light chain sequences are mixed and matched, a heavy chain sequence from a particular heavy chain/light chain pairing is preferably replaced with a structurally similar full-length heavy chain sequence, and a light chain sequence from a particular full-length heavy chain/full-length light chain pairing is preferably replaced with a structurally similar light chain sequence.

In an embodiment, an antibody can have heavy and light chain variable regions whose amino acid sequences are homologous to those of the examples described, and the antibody retains the desired specificity of the examples. In an embodiment, the heavy chain variable region comprises an amino acid sequence that is at least 80% homologous to an amino acid sequence selected from the group consisting of SEQ ID NOs: 65-70. In an embodiment, the light chain variable region comprises an amino acid sequence that is at least 80% homologous to an amino acid sequence selected from the group consisting of SEQ ID NOs: 71-76. In another embodiment, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid sequences encoding SEQ ID NOs: 65-70 and 71-76, followed by testing of the encoded altered antibody for retained function using the functional assays described.

The percent homology between two amino acid sequences equals the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for the optimal alignment of the two sequences.

In an embodiment, an anti-human PD-L1 antibody comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences derived from the anti-human PD-L1 mAbs described (e.g., 2C5, 3B8, 8C6, 10H6, 11G7, and 12H2) by mutagenesis, and wherein the anti-human PD-L1 antibody retains the desired specificity and binding affinity of the embodiments disclosed. In an embodiment, one or more of these CDR sequences of the anti-human PD-L1 antibody are derived from conservative sequence modifications of the preferred antibodies disclosed. In an embodiment, one or more of these CDR sequences of the anti-human PD-L1 are conservatively modified variants of the preferred antibodies disclosed.

The term “conservative sequence modifications” or “conservative sequence modification” refers to amino acid modifications that do not substantially affect or alter the binding characteristics of the antibody containing the amino acid sequence. Such conservative modifications include amino acid substitutions, additions, and deletions. Modifications can be introduced into an antibody disclosed by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of the embodiments disclosed can be replaced with other amino acid residues from the same side chain family and the altered antibody can be tested for retained function (i.e., the functions set forth in (c) through (l) above) using the functional assays described.

The term “conservatively modified variant” or “conservatively modified variants” include individual substitutions, deletions, or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the embodiments herein. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). In some embodiments, the term “conservative sequence modifications” are used to refer to amino acid modifications that do not substantially affect or alter the binding.

Methods

1. Generation, isolation, and purification of rabbit monoclonal antibodies against the extracellular domain of human PD-L1.

The rabbit anti-human PD-L1 mAbs can be generated by a variety of techniques, including monoclonal antibody methodology, e.g., the somatic cell hybridization technique and other techniques including but not limited to viral or oncogenic transformation of B lymphocytes. In an embodiment, the rabbit anti-human PD-L1 mAbs can also be generated by single B cell-based technology.

Immunization protocols and techniques for isolation of immunized splenocytes for fusion are known in the art. Briefly, the extracellular domain of human PD-L1 protein was the immunogen to stimulate an immune response in New Zealand White rabbits. The PD-L1-specific B cells from immunized rabbits were isolated from PBMCs and secondary lymphoid tissues. Candidate IgG genes were amplified from PD-L1-specific B cells for recombinant expression of rabbit mAbs. The binding and specificity of mAb candidates were further validated by direct antigen ELISA. mAbs that can recognize human PD-L1 were further characterized using capture ELISA and sandwich ELISA for assay development.

The rabbit monoclonal antibodies can be isolated and purified by a variety of techniques. In an embodiment, rabbit monoclonal antibodies can be isolated from the culture supernatant from mammalian cells transfected with rabbit antibody genes, subsequently purified by Protein A affinity chromatography. Purity and function of purified rabbit monoclonal antibodies can be verified by SDS-PAGE and ELISA respectively.

2. Preparation of conjugated rabbit monoclonal antibodies against human PD-L1. Rabbit monoclonal antibodies were biotinylated using Pierce EZ-Link Sulfo-NHS-Biotin in accordance with manufacturer manual. Briefly, rabbit monoclonal antibodies in 1×PBS, pH7.4 were incubated with Sulfo-NHS-Biotin for 30 minutes at room temperature.

3. Direct ELISA with rabbit monoclonal antibodies against human PD-L1 or PD-L2. Human PD-L1 and PD-L2 were coated on high binding ELISA plates at 4° C. overnight in 1×PBS, pH7.4. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with rabbit anti-PD-L1 monoclonal antibodies for 1 hour at room temperature, followed by washing and incubation with goat anti-rabbit IgG antibody conjugated with HRP. After a final wash, plated were developed using colorimetric reaction catalyzed by HRP to see if a monoclonal antibody can or cannot bind to PD-L1 and PD-L2.

4. Capture ELISA with rabbit monoclonal antibodies against human PD-L1. Anti-rabbit IgG Fc antibodies were coated on high binding ELISA plates at 4° C. overnight in 1×PBS, pH7.4. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with rabbit anti-PD-L1 monoclonal antibodies for 1 hour at room temperature, followed by washing and incubation with biotinylated PD-L1 protein serial diluted at 3 fold. The plates were further incubated with streptavidin conjugated to HRP. After a final wash, plates were developed using colorimetric reaction catalyzed by HRP to see if a monoclonal antibody can or cannot capture PD-L1.

5. Sandwich ELISA with rabbit monoclonal antibodies against human PD-L1. Capture antibodies were coated on high binding ELISA plates at 4° C. overnight in bicarbonate buffer. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with human PD-L1 protein serial diluted in 3 folds for 1 hour at room temperature, followed by washing and incubation with biotinylated anti-PD-L1 detection monoclonal antibodies. The plates were further incubated with streptavidin conjugated to HRP. After a final wash, plates were developed using colorimetric reaction catalyzed by HRP to see if a pair of capture and detection monoclonal antibodies can or cannot bind to PD-L1 simultaneously.

The term “a”, “an”, or “the” cover both the singular and the plural reference, unless the context clearly dictates otherwise. The terms “comprising”, “having”, “including”, and “containing” are open-ended terms, which means “including but not limited to”, unless otherwise indicated.

While the disclosure has been illustrated and described in typical embodiments, it is not intended to be limited to the details shown, since various modifications and substitutions can be made without departing from the present disclosure. As such, further modifications and equivalents of the disclosure herein disclosed may occur to persons having ordinary skills in the art using no more than routine experimentation. Such modifications and equivalents of the disclosure herein encompass nuclear acid sequences encoding the amino acid sequences disclosed.

### ASPECTS

Aspect 1. An antibody comprising:

(a) a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, and conservative modifications thereof;

(b) a V_{H} CDR2 selected from the group consisting SEQ ID NO: 7, SEQ ID NO: 8, SEQ ID NO: 9, SEQ ID NO: 10, SEQ ID NO: 11, SEQ ID NO: 12, and conservative modifications thereof;

(c) a V_{H} CDR3 selected from the group consisting of SEQ ID NO: 13, SEQ ID NO: 14, SEQ ID NO: 15, SEQ ID NO: 16, SEQ ID NO: 17, SEQ ID NO: 18, and conservative modifications thereof;

(d) a V_{L} CDR1 selected from the group consisting SEQ ID NO: 19, SEQ ID NO: 20, SEQ ID NO: 21, SEQ ID NO: 22, SEQ ID NO: 23, SEQ ID NO: 24, and conservative modifications thereof;

(e) a V_{L} CDR2 selected from the group consisting of SEQ ID NO: 25, SEQ ID NO: 26, SEQ ID NO: 27, SEQ ID NO: 28, SEQ ID NO: 29, SEQ ID NO: 30, and conservative modifications thereof; and

(f) a V_{L} CDR3 selected from the group consisting of SEQ ID NO: 31, SEQ ID NO: 32, SEQ ID NO: 33, SEQ ID NO: 34, SEQ ID NO: 35, SEQ ID NO: 36, and conservative modifications thereof.

Aspect 2. The antibody of aspect 1, wherein

(a) the V_{H} CDR1 includes the amino acid sequence of SEQ ID NO: 1 or a conservative modification thereof;

(b) the V_{H} CDR2 includes the amino acid sequence of SEQ ID NO: 7 or a conservative modification thereof;

(c) the V_{L} CDR3 includes the amino acid sequence of SEQ ID NO: 13 or a conservative modification thereof;

(d) the V_{L} CDR1 includes the amino acid sequence of SEQ ID NO: 19 or a conservative modification thereof;

(e) the V_{L} CDR2 includes the amino acid sequence of SEQ ID NO: 25 or a conservative modification thereof; and

(f) the V_{L} CDR3 includes the amino acid sequence of SEQ ID NO: 31 or a conservative modification thereof.

Aspect 3. The antibody of aspect 1, wherein

(a) the V_{H} CDR1 includes the amino acid sequence of SEQ ID NO: 2 or a conservative modification thereof;

(b) the V_{H} CDR2 includes the amino acid sequence of SEQ ID NO: 8 or a conservative modification thereof;

(c) the V_{H} CDR3 includes the amino acid sequence of SEQ ID NO: 14 or a conservative modification thereof;

(d) the V_{L} CDR1 includes the amino acid sequence of SEQ ID NO: 20 or a conservative modification thereof;

(e) the V_{L} CDR2 includes the amino acid sequence of SEQ ID NO: 26 or a conservative modification thereof; and

(f) the V_{L} CDR3 includes the amino acid sequence of SEQ ID NO: 32 or a conservative modification thereof.

Aspect 4. The antibody of aspect 1, wherein

(a) the V_{H} CDR1 includes the amino acid sequence of SEQ ID NO: 3 or a conservative modification thereof;

(b) the V_{H} CDR2 includes the amino acid sequence of SEQ ID NO: 9 or a conservative modification thereof;

(c) the V_{H} CDR3 includes the amino acid sequence of SEQ ID NO: 15 or a conservative modification thereof;

(d) the V_{L} CDR1 includes the amino acid sequence of SEQ ID NO: 21 or a conservative modification thereof;

(e) the V_{L} CDR2 includes the amino acid sequence of SEQ ID NO: 27 or a conservative modification thereof; and

(f) the V_{L} CDR3 includes the amino acid sequence of SEQ ID NO: 33 or a conservative modification thereof.

Aspect 5. The antibody of aspect 1, wherein

(a) the V_{H} CDR1 includes the amino acid sequence of SEQ ID NO: 4 or a conservative modification thereof;

(b) the V_{H} CDR2 includes the amino acid sequence of SEQ ID NO: 10 or a conservative modification thereof;

(c) the V_{H} CDR3 includes the amino acid sequence of SEQ ID NO: 16 or a conservative modification thereof;

(d) the V_{L} CDR1 includes the amino acid sequence of SEQ ID NO: 22 or a conservative modification thereof;

(e) the V_{L} CDR2 includes the amino acid sequence of SEQ ID NO: 28 or a conservative modification thereof; and

(f) the V_{L} CDR3 includes the amino acid sequence of SEQ ID NO: 34 or a conservative modification thereof.

Aspect 6. The antibody of aspect 1, wherein

(a) the V_{H} CDR1 includes the amino acid sequence of SEQ ID NO: 5 or a conservative modification thereof;

(b) the V_{H} CDR2 includes the amino acid sequence of SEQ ID NO: 11 or a conservative modification thereof;

(c) the V_{H} CDR3 includes the amino acid sequence of SEQ ID NO: 17 or a conservative modification thereof;

(d) the V_{L} CDR1 includes the amino acid sequence of SEQ ID NO: 23 or a conservative modification thereof;

(e) the V_{L} CDR2 includes the amino acid sequence of SEQ ID NO: 29 or a conservative modification thereof; and

(f) the V_{L} CDR3 includes the amino acid sequence of SEQ ID NO: 35 or a conservative modification thereof.

Aspect 7. The antibody of aspect 1, wherein

(a) the V_{H} CDR1 includes the amino acid sequence of SEQ ID NO: 6 or a conservative modification thereof;

(b) the V_{H} CDR2 includes the amino acid sequence of SEQ ID NO: 12 or a conservative modification thereof;

(c) the V_{H} CDR3 includes the amino acid sequence of SEQ ID NO: 18 or a conservative modification thereof;

(d) the V_{L} CDR1 includes the amino acid sequence of SEQ ID NO: 24 or a conservative modification thereof;

(e) the V_{L} CDR2 includes the amino acid sequence of SEQ ID NO: 30 or a conservative modification thereof; and

(f) the V_{L} CDR3 includes the amino acid sequence of SEQ ID NO: 36 or a conservative modification thereof.

Aspect 8. The antibody as in any one of aspects 1-7, wherein the antibody further comprises

(a) a V_{H} FR1 includes an amino acid sequence selected from the group consisting of SEQ ID NO: 37, SEQ ID NO: 38, SEQ ID NO: 39, and SEQ ID NO: 40;

(b) a V_{H} FR2 includes an amino acid sequence of SEQ ID NO: 41 and SEQ ID NO: 42;

(c) a V_{H} FR3 includes an amino acid sequence selected from the group consisting of SEQ ID NO: 43, SEQ ID NO: 44, SEQ ID NO: 45, SEQ ID NO: 46, and SEQ ID NO: 47;

(d) a V_{H} FR4 includes an amino acid sequence selected from the group consisting of SEQ ID NO: 48 and SEQ ID NO: 49;

(e) a V_{L} FR1 includes an amino acid sequence selected from the group consisting of SEQ ID NO: 50, SEQ ID NO: 51, and SEQ ID NO: 52, SEQ ID NO: 53, and SEQ ID NO: 54;

(f) a V_{L} FR2 includes an amino acid sequence of selected from the group consisting SEQ ID NO: 55 and SEQ ID NO: 56;

(e) a V_{L} FR3 includes an amino acid sequence selected from the group consisting of SEQ ID NO: 57, SEQ ID NO: 58, SEQ ID NO: 59, SEQ ID NO: 60, SEQ ID NO: 61, and SEQ ID NO: 62; and

(g) a V_{L} FR4 includes an amino acid sequence selected from the group consisting of SEQ ID NO: 63 and SEQ ID NO: 64.

Aspect 9. An antibody, comprising:

(a) a V_{H} includes an amino acid sequence selected from the group consisting of SEQ ID NO: 65, SEQ ID NO: 66, SEQ ID NO: 67, SEQ ID NO: 68, SEQ ID NO: 69, SEQ ID NO: 70, and conservative modifications thereof; and

(b) a V_{H} includes an amino acid sequence selected from the group consisting of SEQ ID NO: 71, SEQ ID NO: 72, SEQ ID NO: 73, SEQ ID NO: 74, SEQ ID NO: 75, SEQ ID NO: 76, and conservative modifications thereof;

Aspect 10. The antibody of aspect 9, wherein the V_{H} includes an amino acid sequence of SEQ ID NO: 65, and the V_{L} includes an amino acid sequence of SEQ ID NO:71.
Aspect 11. The antibody of aspect 9, wherein the V_{H} includes an amino acid sequence of SEQ ID NO: 66, and the V_{L} includes an amino acid sequence of SEQ ID NO:72.
Aspect 12. The antibody of aspect 9, wherein the V_{H} includes an amino acid sequence of SEQ ID NO: 67, and the V_{L} includes an amino acid sequence of SEQ ID NO:73.
Aspect 13. The antibody of aspect 9, wherein the V_{H} includes an amino acid sequence of SEQ ID NO: 68, and the V_{L} includes an amino acid sequence of SEQ ID NO:74.
Aspect 14. The antibody as in any one of aspect 9, wherein the V_{H} includes an amino acid sequence of SEQ ID NO: 69, and V_{L} includes an amino acid sequence of SEQ ID NO:75.
Aspect 15. The antibody of aspect 9, wherein the V_{H} includes an amino acid sequence of SEQ ID NO: 70, and the V_{L} includes an amino acid sequence of SEQ ID NO: 76.
Aspect 16. An antibody, comprising a Fab fragment including a heavy chain and a light chain,

wherein the heavy chain includes an amino acid sequence selected from the group consisting of SEQ ID NO: 77, SEQ ID NO: 78, SEQ ID NO: 79, SEQ ID NO: 80, SEQ ID NO: 81, SEQ ID NO: 82, and conservative modifications thereof, and

the light chain includes an amino acid sequence selected from the group consisting of SEQ ID NO: 83, SEQ ID NO: 84, SEQ ID NO: 85, SEQ ID NO: 86, SEQ ID NO: 87, SEQ ID NO: 88, and conservative modifications thereof;

Aspect 17. The antibody of aspect 16, wherein the heavy chain includes an amino acid sequence of SEQ ID NO: 77, and the light chain includes an amino acid sequence of SEQ ID NO: 83.
Aspect 18. The antibody of aspect 9, wherein the heavy chain includes an amino acid sequence of SEQ ID NO: 78, and the light chain includes an amino acid sequence of SEQ ID NO: 84.
Aspect 19. The antibody of aspect 9, wherein the heavy chain includes an amino acid sequence of SEQ ID NO: 79, and the light chain includes an amino acid sequence of SEQ ID NO: 85.
Aspect 20. The antibody of aspect 9, wherein the heavy chain includes an amino acid sequence of SEQ ID NO: 80, and the light chain includes an amino acid sequence of SEQ ID NO: 86.
Aspect 21. The antibody of aspect 9, wherein the heavy chain includes an amino acid sequence of SEQ ID NO: 81, and the light chain includes an amino acid sequence of SEQ ID NO: 87.
Aspect 22. The antibody of aspect 9, wherein the heavy chain includes an amino acid sequence of SEQ ID NO: 82, and the light chain includes an amino acid sequence of SEQ ID NO: 88.
Aspect 23. The antibody as in any one of aspects 1-22, further comprising an Fc fragment that has an amino acid sequence selected from the group consisting of SEQ ID NO: 89, SEQ ID NO: 90, SEQ ID NO: 91, and conservative modifications thereof.
Aspect 24. The antibody as in any one of aspects 1-23, wherein the antibody further comprises a covalently or non-covalently attached conjugate.
Aspect 25. The antibody of aspect 24, wherein the conjugate includes an enzyme, a fluorophore, a biotin, or a streptavidin, or a combination thereof.
Aspect 26. The antibody as in any one of aspects 1-25, wherein the antibody is a monoclonal antibody or a polyclonal antibody, or a combination thereof.
Aspect 27. The antibody as in any one of aspects 1-26, wherein the antibody is a humanized or chimeric antibody.
Aspect 28. The antibody as in any one of aspects 1-27, wherein the antibody is an anti-human PD-L1 antibody.
Aspect 29. The antibody as in any one of aspects 1-28, wherein the antibody has minimal or no detectable cross-reactivity to PD-L2.s
Aspect 30. The antibody as in any one of aspects 1-29, wherein the CDRs have homology in a range from 47.4% to 100%.
Aspect 31. The antibody as in any one of aspects 1-30, wherein the FRs have homology in a range from 55.6% to 100%.
Aspect 32. The antibody as in any one of aspects 1-31, wherein the antibody contains one or more of rabbit germline CDRs.
Aspect 33. An ELISA kit for detection of expression of human PD-L1, comprising the antibody as in any one of aspects 1-32.

Aspect 34. A method for detecting human PD-L1, comprising:
adding a first antibody that is the antibody as in any one of aspects 1-30.
   Aspect 35. The method of aspect 34, wherein the method is a direct ELISA.
   Aspect 36. The method of aspect 34, wherein the method is a capture ELISA.
   Aspect 37. The method of aspect 34, wherein the method is a sandwich ELISA.
   Aspect 38. The method for detecting human PD-L1, further comprising:

adding a second antibody, wherein the first antibody captures an extracellular domain of human PD-L1, and the second antibody includes a conjugate for detection,

the first antibody is different from the second antibody,

the first and second antibodies are each selected from the group consisting of 3B8, 2C5, and 8C6.

We claim:

1. An antibody comprising:
(a) a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, and conservative modifications thereof;
(b) a V_{H} CDR2 selected from the group consisting SEQ ID NO: 7, SEQ ID NO: 8, SEQ ID NO: 9, SEQ ID NO: 10, SEQ ID NO: 11, SEQ ID NO: 12, and conservative modifications thereof;
(c) a V_{H} CDR3 selected from the group consisting of SEQ ID NO: 13, SEQ ID NO: 14, SEQ ID NO: 15, SEQ ID NO: 16, SEQ ID NO: 17, SEQ ID NO: 18, and conservative modifications thereof;
(d) a V_{L} CDR1 selected from the group consisting SEQ ID NO: 19, SEQ ID NO: 20, SEQ ID NO: 21, SEQ ID NO: 22, SEQ ID NO: 23, SEQ ID NO: 24, and conservative modifications thereof;
(e) a V_{L} CDR2 selected from the group consisting of SEQ ID NO: 25, SEQ ID NO: 26, SEQ ID NO: 27, SEQ ID NO: 28, SEQ ID NO: 29, SEQ ID NO: 30, and conservative modifications thereof; and
(f) a V_{L} CDR3 selected from the group consisting of SEQ ID NO: 31, SEQ ID NO: 32, SEQ ID NO: 33, SEQ ID NO: 34, SEQ ID NO: 35, SEQ ID NO: 36, and conservative modifications thereof.

2. The antibody of claim 1, wherein the antibody comprises
(a) the V_{H} CDR1 includes the amino acid sequence of SEQ ID NO: 1 or a conservative modification thereof, the V_{H} CDR2 includes the amino acid sequence of SEQ ID NO: 7 or a conservative modification thereof, the V_{H} CDR3 includes the amino acid sequence of SEQ ID NO: 13 or a conservative modification thereof, the V_{L} CDR1 includes the amino acid sequence of SEQ ID NO: 19 or a conservative modification thereof, the V_{L} CDR2 includes the amino acid sequence of SEQ ID NO: 25 or a conservative modification thereof, and the V_{L} CDR3 includes the amino acid sequence of SEQ ID NO: 31 or a conservative modification thereof; or
(b) the V_{H} CDR1 includes the amino acid sequence of SEQ ID NO: 2 or a conservative modification thereof, the V_{H} CDR2 includes the amino acid sequence of SEQ ID NO: 8 or a conservative modification thereof, the V_{H} CDR3 includes the amino acid sequence of SEQ ID NO: 14 or a conservative modification thereof; the V_{L} CDR1 includes the amino acid sequence of SEQ ID NO: 20 or a conservative modification thereof; the V_{L} CDR2 includes the amino acid sequence of SEQ ID NO: 26 or a conservative modification thereof; and the V_{L} CDR3 includes the amino acid sequence of SEQ ID NO: 32 or a conservative modification thereof; or
(c) the V_{H} CDR1 includes the amino acid sequence of SEQ ID NO: 3 or a conservative modification thereof, the V_{H} CDR2 includes the amino acid sequence of SEQ ID NO: 9 or a conservative modification thereof, the V_{H} CDR3 includes the amino acid sequence of SEQ ID NO: 15 or a conservative modification thereof, the V_{L} CDR1 includes the amino acid sequence of SEQ ID NO: 21 or a conservative modification thereof, the V_{L} CDR2 includes the amino acid sequence of SEQ ID NO: 27 or a conservative modification thereof, and the V_{L} CDR3 includes the amino acid sequence of SEQ ID NO: 33 or a conservative modification thereof; or
(d) the V_{H} CDR1 includes the amino acid sequence of SEQ ID NO: 4 or a conservative modification thereof, the V_{H} CDR2 includes the amino acid sequence of SEQ ID NO: 10 or a conservative modification thereof, the V_{H} CDR3 includes the amino acid sequence of SEQ ID NO: 16 or a conservative modification thereof, the V_{L} CDR1 includes the amino acid sequence of SEQ ID NO: 22 or a conservative modification thereof, the V_{L} CDR2 includes the amino acid sequence of SEQ ID NO: 28 or a conservative modification thereof, and the V_{L} CDR3 includes the amino acid sequence of SEQ ID NO: 34 or a conservative modification thereof; or
(e) the V_{H} CDR1 includes the amino acid sequence of SEQ ID NO: 5 or a conservative modification thereof, the V_{H} CDR2 includes the amino acid sequence of SEQ ID NO: 11 or a conservative modification thereof, the V_{H} CDR3 includes the amino acid sequence of SEQ ID NO: 17 or a conservative modification thereof, the V_{L} CDR1 includes the amino acid sequence of SEQ ID NO: 23 or a conservative modification thereof, the V_{L} CDR2 includes the amino acid sequence of SEQ ID NO: 29 or a conservative modification thereof, and the V_{L} CDR3 includes the amino acid sequence of SEQ ID NO: 35 or a conservative modification thereof; or
(f) the V_{H} CDR1 includes the amino acid sequence of SEQ ID NO: 6 or a conservative modification thereof, the V_{H} CDR2 includes the amino acid sequence of SEQ ID NO: 12 or a conservative modification thereof, the V_{H} CDR3 includes the amino acid sequence of SEQ ID NO: 18 or a conservative modification thereof, the V_{L} CDR1 includes the amino acid sequence of SEQ ID NO: 24 or a conservative modification thereof, the V_{L} CDR2 includes the amino acid sequence of SEQ ID NO: 30 or a conservative modification thereof, the V_{L} CDR3 includes the amino acid sequence of SEQ ID NO: 36 or a conservative modification thereof.

3. The antibody of claim 1, wherein the antibody has no substantially detectable or no detectable cross-reactivity to PD-L2.

4. The antibody of claim 1, wherein the antibody is a rabbit monoclonal or polyclonal antibody or a fragment thereof.

5. The antibody of claim 1, wherein the CDRs have homology in a range from 47.4% to 100%.

6. The antibody of claim 1, further comprising FRs, wherein the FRs comprise:
(a) a V_{H} FR1 includes an amino acid sequence selected from the group consisting of SEQ ID NO: 37, SEQ ID NO: 38, SEQ ID NO: 39, and SEQ ID NO: 40,
(b) a V_{H} FR2 includes an amino acid sequence of SEQ ID NO: 41 and SEQ ID NO: 42;
(c) a V_{H} FR3 includes an amino acid sequence selected from the group consisting of SEQ ID NO: 43, SEQ ID NO: 44, SEQ ID NO: 45, SEQ ID NO: 46, and SEQ ID NO: 47;
(d) a V_{H} FR4 includes an amino acid sequence selected from the group consisting of SEQ ID NO: 48 and SEQ ID NO: 49;
(e) a V_{L} FR1 includes an amino acid sequence selected from the group consisting of SEQ ID NO: 50, SEQ ID NO: 51, and SEQ ID NO: 52, SEQ ID NO: 53, and SEQ ID NO: 54;
(f) a V_{L} FR2 includes an amino acid sequence of selected from the group consisting SEQ ID NO: 55 and SEQ ID NO: 56;
(e) a V_{L} FR3 includes an amino acid sequence selected from the group consisting of SEQ ID NO: 57, SEQ ID NO: 58, SEQ ID NO: 59, SEQ ID NO: 60, SEQ ID NO: 61, and SEQ ID NO: 62; and
(g) a V_{L} FR4 includes an amino acid sequence from the group consisting of SEQ ID NO: 63 and SEQ ID NO: 64.

7. The antibody of claim 6, wherein the FRs have homology in a range from 55.6% to 100.0%

8. An antibody, comprising:
(a) a V_{H} includes an amino acid sequence selected from the group consisting of SEQ ID NO: 65, SEQ ID NO: 66, SEQ ID NO: 67, SEQ ID NO: 68, SEQ ID NO: 69, SEQ ID NO: 70, and conservative modifications thereof; and
(b) a V_{L} includes an amino acid sequence selected from the group consisting of 71, SEQ ID NO: 72, SEQ ID NO: 73, SEQ ID NO: 74, SEQ ID NO: 75, SEQ ID NO: 76, and conservative modifications thereof.

9. The antibody of claim 8, wherein
(a) the V_{H} includes the amino acid sequence of SEQ ID NO.: 65 and the V_{L} includes the amino acid sequence of SEQ ID NO.: 71; or
(b) the V_{H} includes the amino acid sequence of SEQ ID NO.: 66 and the V_{L} includes the amino acid sequence of SEQ ID NO.: 72; or
(c) the V_{H} includes the amino acid sequence of SEQ ID NO.: 67 and the V_{L} includes the amino acid sequence of SEQ ID NO.: 73; or
(d) the V_{H} includes the amino acid sequence of SEQ ID NO.: 68 and the V_{L} includes the amino acid sequence of SEQ ID NO.: 74; or
(e) the V_{H} includes the amino acid sequence of SEQ ID NO.: 69 and the V_{L} includes the amino acid sequence of SEQ ID NO.: 75; or
(f) the V_{H} includes the amino acid sequence of SEQ ID NO.: 70 and the V_{L} includes the amino acid sequence of SEQ ID NO.: 76;

10. The antibody of claim 1, wherein the antibody further comprises a covalently or non-covalently attached conjugate.

11. The antibody of claim 9, wherein the conjugate includes an enzyme, a fluorescence protein, a fluorophore, a biotin, or a streptavidin.

12. The antibody of claim 9, wherein the enzyme includes HRP.

13. The antibody of claim 1, wherein the antibody is a humanized or chimeric antibody.

14. A kit for detecting an expression of human PD-L1 comprising the antibody of claim 1.

15. A method for detecting human PD-L1, comprising:
adding the antibody of claim 1.

16. The method of claim 15, wherein the method is a direct ELISA, and the antibody detects human PD-L1 attached to a supportive surface.

17. The method of claim 15, wherein the method is a capture ELISA.

18. The method of claim 15, wherein the method is a sandwich ELISA.

19. The method for detecting human PD-L1, further comprising:
adding a second antibody, wherein the antibody captures an extracellular domain of human PD-L1, and the second antibody includes a conjugate for detection,
the antibody is different from the second antibody,
the antibody and the second antibody are each selected from the group consisting of 3B8, 2C5, and 8C6.


SEQUENCE LISTING (91 sequences)

SEQ ID NO: 1 - protein, 9 aa, rabbit
FSLNNDNVG

SEQ ID NO: 2 - protein, 10 aa, rabbit
FSFSNNAYMC

SEQ ID NO: 3 - protein, 9 aa, rabbit
FDISRHHMC

SEQ ID NO: 4 - protein, 10 aa, rabbit
FSFSSIYYLC

SEQ ID NO: 5 - protein, 9 aa, rabbit
IDLSSYDMT

SEQ ID NO: 6 - protein, 10 aa, rabbit
FSFSSSYYMC

SEQ ID NO: 7 - protein, 18 aa, rabbit
WVGLIDILGSSYYASWAK

SEQ ID NO: 8 - protein, 19 aa, rabbit
LIACIYSRYGNTHYASWAK

SEQ ID NO: 9 - protein, 18 aa, rabbit
YIGFINPPGSAYYANWAK

SEQ ID NO: 10 - protein, 20 aa, rabbit
WIACIYGGNSDNTWYASWAK

SEQ ID NO: 11 - protein, 18 aa, rabbit
YIGYISYFDKTYYATWAK

SEQ ID NO: 12 - protein, 20 aa, rabbit
WIACIWTSNGGRTYYANWAK

SEQ ID NO: 13 - protein, 13 aa, rabbit
YFCAREGDGSFDP

SEQ ID NO: 14 - protein, 19 aa, rabbit
YFCARGASGSSDYSFYFNL

SEQ ID NO: 15 - protein, 14 aa, rabbit
YFCARWRFDDYGDL

SEQ ID NO: 16 - protein, 16 aa, rabbit
YFCARGYDIYGYAINL

SEQ ID NO: 17 - protein, 15 aa, rabbit
YFCVRDRPDGITANL

SEQ ID NO: 18 - protein, 16 aa, rabbit
YFCARDDDTGSGYYNL

SEQ ID NO: 19 - protein, 11 aa, rabbit
QNVYNKKNLAW

SEQ ID NO: 20 - protein, 9 aa, rabbit
QSISSWLAW

SEQ ID NO: 21 - protein, 9 aa, rabbit
ESIASYLAW

SEQ ID NO: 22 - protein, 9 aa, rabbit
QSIGGYLAW

SEQ ID NO: 23 - protein, 11 aa, rabbit
HNVYSDNRLSW

SEQ ID NO: 24 - protein, 9 aa, rabbit
ESISSWLAW

SEQ ID NO: 25 - protein, 11 aa, rabbit
LIYKASTLASG

SEQ ID NO: 26 - protein, 11 aa, rabbit
LIYDAYDLASG

SEQ ID NO: 27 - protein, 11 aa, rabbit
LIYKASTLASG

SEQ ID NO: 28 - protein, 11 aa, rabbit
LIYRASTLESG

SEQ ID NO: 29 - protein, 11 aa, rabbit
LIYWASILASG

SEQ ID NO: 30 - protein, 11 aa, rabbit
LIYKASTLASG

SEQ ID NO: 31 - protein, 9 aa, rabbit
GDYIGSRAF

SEQ ID NO: 32 - protein, 12 aa, rabbit
QGYSSENIDNSF

SEQ ID NO: 33 - protein, 12 aa, rabbit
QGYSATNIDNAF

SEQ ID NO: 34 - protein, 15 aa, rabbit
CTYDSSNSRIYPNVF

SEQ ID NO: 35 - protein, 10 aa, rabbit
GGYSGNIYTF

SEQ ID NO: 36 - protein, 12 aa, rabbit
HGYYFGSVANTF

SEQ ID NO: 37 - protein, 25 aa, rabbit
QSVEESGGRLVTPGTPLTLTCTVSG

SEQ ID NO: 38 - protein, 25 aa, rabbit
QSLEESGGDLVKPGASRTLTCTASG

SEQ ID NO: 39 - protein, 25 aa, rabbit
QSVEESGGRLVTPGTPLTLTCTASG

SEQ ID NO: 40 - protein, 26 aa, rabbit
QEQLVESGGGLVQPEGSLTLTCTASG

SEQ ID NO: 41 - protein, 11 aa, rabbit
WVRQAPGKGLE

SEQ ID NO: 42 - protein, 11 aa, rabbit
WVRQAPGEGLE

SEQ ID NO: 43 - protein, 26 aa, rabbit
GRFTISKTSNTVDLKITSPTIEDTAT

SEQ ID NO: 44 - protein, 27 aa, rabbit
GRFTISKTSSTTVTLQMTSLTAADTAT

SEQ ID NO: 45 - protein, 26 aa, rabbit
GRFTISRTSTTVALEMTSLTTEDTAT

SEQ ID NO: 46 - protein, 26 aa, rabbit
GRFTISKTSTTVDLKMTSLTTEDTAT

SEQ ID NO: 47 - protein, 27 aa, rabbit
GRFTISETSSTTVTLQMTSLTAADTAT

SEQ ID NO: 48 - protein, 11 aa, rabbit
WGPGTLVTVSS

SEQ ID NO: 49 - protein, 11 aa, rabbit
WGQGTLVTVSS

SEQ ID NO: 50 - protein, 26 aa, rabbit
AQVLTQTPSPVSAAVGGTVTINCQAS

SEQ ID NO: 51 - protein, 26 aa, rabbit
AYDMTQTPASVEVAVGGTVTIKCQAS

SEQ ID NO: 52 - protein, 26 aa, rabbit
DVVMTQTPASVEAAVGGTVTIKCQAS

SEQ ID NO: 53 - protein, 26 aa, rabbit
AQLPTQTASPVSAAVGGTVTINCQSS

SEQ ID NO: 54 - protein, 27 aa, rabbit
AQIVMTQTASPVSAAVGGTVTINCQAS

SEQ ID NO: 55 - protein, 11 aa, rabbit
YQQKLGQPPKL

SEQ ID NO: 56 - protein, 11 aa, rabbit
YQQKPGQPPKL

SEQ ID NO: 57 - protein, 32 aa, rabbit
VPSRFSGSGSGTQFTLTISDLECDDAATYYCQ

SEQ ID NO: 58 - protein, 32 aa, rabbit
VPSRFKGSGSGTQFTLTISGVECTDAATYYCQ

SEQ ID NO: 59 - protein, 32 aa, rabbit
VSSRFRGSGSGTEFTLTISGVECADAATYYCQ

SEQ ID NO: 60 - protein, 32 aa, rabbit
VASRFKGSGSGTEFTLTISDLECADAATYYCQ

SEQ ID NO: 61 - protein, 32 aa, rabbit
VPSRFKGSGSGTQFTLTITDLECDDAATYYCA

SEQ ID NO: 62 - protein, 32 aa, rabbit
VPSRFKGSGSGTEFTLTISDLECADAATYYCQ

SEQ ID NO: 63 - protein, 9 aa, rabbit
GGGTEVVVK

SEQ ID NO: 64 - protein, 9 aa, rabbit
GGGTDVVVK

SEQ ID NO: 65 - protein, 113 aa, rabbit
QSVEESGGRLVTPGTPLTLTCTVSGFSLNNDNVGWVRQAPGKGLEWVGLIDILGSSYYASWAKGRFTISKTSNTVDLKITSPTIEDTATYFCAREGDGSFDPWGPGTLVTVSS

SEQ ID NO: 66 - protein, 122 aa, rabbit
QSLEESGGDLVKPGASRTLTCTASGFSFSNNAYMCWVRQAPGKGLELIACIYSRYGNTHYASWAKGRFTISKTSSTTVTLQMTSLTAADTATYFCARGASGSSDYSFYFNLWGPGTLVTVSS

SEQ ID NO: 67 - protein, 114 aa, rabbit
QSVEESGGRLVTPGTPLTLTCTASGFDISRHHMCWVRQAPGEGLEYIGFINPPGSAYYANWAKGRFTISRTSTTVALEMTSLTTEDTATYFCARWRFDDYGDLWGQGTLVTVSS

SEQ ID NO: 68 - protein, 121 aa, rabbit
QEQLVESGGGLVQPEGSLTLTCTASGFSFSSIYYLCWVRQAPGKGLEWIACIYGGNSDNTWYASWAKGRFTISKTSSTTVTLQMTSLTAADTATYFCARGYDIYGYAINLWGPGTLVTVSS

SEQ ID NO: 69 - protein, 115 aa, rabbit
QSVEESGGRLVTPGTPLTLTCTVSGIDLSSYDMTWVRQAPGKGLEYIGYISYFDKTYYATWAKGRFTISKTSTTVDLKMTSLTTEDTATYFCVRDRPDGITANLWGQGTLVTVSS

SEQ ID NO: 70 - protein, 121 aa, rabbit
QEQLVESGGGLVQPEGSLTLTCTASGFSFSSSYYMCWVRQAPGKGLEWIACIWTSNGGRTYYANWAKGRFTISETSSTTVTLQMTSLTAADTATYFCARDDDTGSGYYNLWGPGTLVTVSS

SEQ ID NO: 71 - protein, 109 aa, rabbit
AQVLTQTPSPVSAAVGGTVTINCQASQNVYNKKNLAWYQQKLGQPPKLLIYKASTLASGVPSRFSGSGSGTQFTLTISDLECDDAATYYCQGDYIGSRAFGGGTEVVVK

SEQ ID NO: 72 - protein, 110 aa, rabbit
AYDMTQTPASVEVAVGGTVTIKCQASQSISSWLAWYQQKPGQPPKLLIYDAYDLASGVPSRFKGSGSGTQFTLTISGVECTDAATYYCQQGYSSENIDNSFGGGTDVVVK

SEQ ID NO: 73 - protein, 110 aa, rabbit
AYDMTQTPASVEVAVGGTVTIKCQASESIASYLAWYQQKPGQPPKLLIYKASTLASGVSSRFRGSGSGTEFTLTISGVECADAATYYCQQGYSATNIDNAFGGGTEVVVK

SEQ ID NO: 74 - protein, 113 aa, rabbit
DVVMTQTPASVEAAVGGTVTIKCQASQSIGGYLAWYQQKPGQPPKLLIYRASTLESGVASRFKGSGSGTEFTLTISDLECADAATYYCQCTYDSSNSRIYPNVFGGGTEVVVK

SEQ ID NO: 75 - protein, 110 aa, rabbit
AQLPTQTASPVSAAVGGTVTINCQSSHNVYSDNRLSWYQQKPGQPPKLLIYWASILASGVPSRFKGSGSGTQFTLTITDLECDDAATYYCAGGYSGNIYTFGGGTEVVVK

SEQ ID NO: 76 - protein, 111 aa, rabbit
AQIVMTQTASPVSAAVGGTVTINCQASESISSWLAWYQQKPGQPPKLLIYKASTLASGVPSRFKGSGSGTEFTLTISDLECADAATYYCQHGYYFGSVANTFGGGTEVVVK

SEQ ID NO: 77 - protein, 208 aa, rabbit
QSVEESGGRLVTPGTPLTLTCTVSGFSLNNDNVGWVRQAPGKGLEWVGLIDILGSSYYASWAKGRFTISKTSNTVDLKITSPTIEDTATYFCAREGDGSFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 78 - protein, 217 aa, rabbit
QSLEESGGDLVKPGASRTLTCTASGFSFSNNAYMCWVRQAPGKGLELIACIYSRYGNTHYASWAKGRFTISKTSSTTVTLQMTSLTAADTATYFCARGASGSSDYSFYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 79 - protein, 209 aa, rabbit
QSVEESGGRLVTPGTPLTLTCTASGFDISRHHMCWVRQAPGEGLEYIGFINPPGSAYYANWAKGRFTISRTSTTVALEMTSLTTEDTATYFCARWRFDDYGDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 80 - protein, 216 aa, rabbit
QEQLVESGGGLVQPEGSLTLTCTASGFSFSSIYYLCWVRQAPGKGLEWIACIYGGNSDNTWYASWAKGRFTISKTSSTTVTLQMTSLTAADTATYFCARGYDIYGYAINLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 81 - protein, 210 aa, rabbit
QSVEESGGRLVTPGTPLTLTCTVSGIDLSSYDMTWVRQAPGKGLEYIGYISYFDKTYYATWAKGRFTISKTSTTVDLKMTSLTTEDTATYFCVRDRPDGITANLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 82 - protein, 216 aa, rabbit
QEQLVESGGGLVQPEGSLTLTCTASGFSFSSSYYMCWVRQAPGKGLEWIACIWTSNGGRTYYANWAKGRFTISETSSTTVTLQMTSLTAADTATYFCARDDDTGSGYYNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 83 - protein, 213 aa, rabbit
AQVLTQTPSPVSAAVGGTVTINCQASQNVYNKKNLAWYQQKLGQPPKLLIYKASTLASGVPSRFSGSGSGTQFTLTISDLECDDAATYYCQGDYIGSRAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 84 - protein, 214 aa, rabbit
AYDMTQTPASVEVAVGGTVTIKCQASQSISSWLAWYQQKPGQPPKLLIYDAYDLASGVPSRFKGSGSGTQFTLTISGVECTDAATYYCQQGYSSENIDNSFGGGTDVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 85 - protein, 214 aa, rabbit
AYDMTQTPASVEVAVGGTVTIKCQASESIASYLAWYQQKPGQPPKLLIYKASTLASGVSSRFRGSGSGTEFTLTISGVECADAATYYCQQGYSATNIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 86 - protein, 217 aa, rabbit
DVVMTQTPASVEAAVGGTVTIKCQASQSIGGYLAWYQQKPGQPPKLLIYRASTLESGVASRFKGSGSGTEFTLTISDLECADAATYYCQCTYDSSNSRIYPNVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 87 - protein, 214 aa, rabbit
AQLPTQTASPVSAAVGGTVTINCQSSHNVYSDNRLSWYQQKPGQPPKLLIYWASILASGVPSRFKGSGSGTQFTLTITDLECDDAATYYCAGGYSGNIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 88 - protein, 215 aa, rabbit
AQIVMTQTASPVSAAVGGTVTINCQASESISSWLAWYQQKPGQPPKLLIYKASTLASGVPSRFKGSGSGTEFTLTISDLECADAATYYCQHGYYFGSVANTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 89 - protein, 228 aa, rabbit
APSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 90 - protein, 228 aa, rabbit
APSTCSKPTCPPPELLGGPSVFIFPPKPKDTLMISRTPEVTCVVVDVSQDDPEVQFTWYINNEQVRTARPPLREQQFNSTIRVVSTLPITHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 91 - protein, 228 aa, rabbit
APSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK